(19) United States
(12) Patent Application Publication
(10) Pub. No.: US 2015/0305333 A1
(43) Pub. Date: Oct. 29, 2015

(54) USE OF GLUCOSAMINE AMIDES AS PLANT GROWTH AND YIELD ENHANCERS

(71) Applicants: PIONEER HI-BRED INTERNATIONAL, INC., Johnston, IA (US); E.I. DU PONT DE NEMOURS & COMPANY, Wilmington, (DE)

(72) Inventors: Keith A. O'Bryan, Johnston, IA (US); France Rochette, Toronto (CA); Subramaniam Sabesan, WILMINGTON, DE (US)

(73) Assignee: PIONEER HI-BRED INTERNATIONAL, INC., Johnston, IA (US)

(21) Appl. No.: 14/439,338

(22) PCT Filed: Oct. 25, 2013
(86) PCT No.: PCT/US13/66815

§ 371 Date: Apr. 29, 2015

Related U.S. Application Data
(60) Provisional application No. 61/719,810, filed on Oct. 29, 2012.

(51) Int. Cl. A01N 43/16; C07H 15/04

(52) U.S. Cl. CPC A01N 43/16; C07H 15/04

(57) ABSTRACT

The invention provides compositions and methods for improving plant growth and crop yield. More specifically, the present invention relates to compositions comprising the glucosamine amide N-palmitoleyl-D-glucosamine (NPG) and other substituted glucosamine compounds. NPG and its substituted analogs may be applied to plant propagating materials, including seeds and other regenerable plant parts, including cuttings, bulbs, rhizomes and tubers. NPG and its analogs may also be applied to foliage or soil either prior to or following planting of plant propagating materials. Such applications may be made alone or in combination with fungicides, insecticides, nematicides and other agricultural agents used to improve plant growth and crop yield. NPG and its analogs can improve the agronomic performance of a variety of crops including barley, canola, corn, potato, soybean and wheat.

### FIELD OF THE INVENTION

The present invention relates to compositions, formulations and methods for improving plant growth and crop yield.

### BACKGROUND

Signaling molecules produced by rhizobia, which include various nitrogen-fixing bacteria, initiate early stage root nodulation in leguminous plants. The resulting symbiotic relationship between the bacteria and plant provides reduced (i.e. “fixed”) nitrogen to the plant and enhances growth and yield. Signaling molecules and rhizobial inoculants are used to increase the productivity of a variety of crops, including soybeans, peanuts, alfalfa, and dry beans.

The use of rhizobial inoculants is, however, constrained by several factors, including variability when produced through biological means. Likewise, individual signaling molecules may be difficult to isolate from mixtures or are not amenable to economical methods of synthesis. Thus, there remains a need for cost-effective alternatives with growth or yield enhancing activity for agricultural applications. The present invention addresses this need.

### SUMMARY OF THE INVENTION

The invention provides compositions and methods for improving plant growth and crop yield. More specifically, the present invention relates to compositions comprising the glucosamine amide N-palmitoleyl-D-glucosamine (NPG) and other substituted glucosamine compounds. NPG and its substituted analogs may be applied to plant propagating materials, including seeds and other regenerable plant parts, including cuttings, bulbs, rhizomes and tubers. NPG and its analogs may also be applied to foliage or soil either prior to or following planting of plant propagating materials. Such applications may be made alone or in combination with fungicides, insecticides, nematicides and other agricultural agents used to improve plant growth and crop yield. NPG and its analogs can improve the agronomic performance of a variety of crops including barley, canola, corn, potato, soybean and wheat.

### DETAILED DESCRIPTION OF THE INVENTION

The invention provides compositions and methods for improving plant growth and crop yield by treating plant propagating materials, foliage or soil with biologically effective amounts of the glucosamine amide N-palmitoleyl-D-glucosamine (NPG) or N-substituted glucosamine compounds of the general Formula (I) herein below

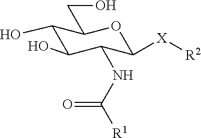

wherein R¹ is C₁-C₂₄ alkyl, C₇-C₂₄ alkaryl, C₆-C₂₄ aryl, C₂-C₂₄ monoalkenyl, C₄-C₂₄ dialkenyl or polyalkenyl, C₂-C₂₄ monoalkynyl, C₄-C₂₄ dialkynyl or polyalkynyl; R² is H, C₁-C₂₄ alkyl, C₇-C₂₄ alkaryl, or C₆-C₂₄ aryl, and X is O or S; in the present compositions R¹ does not terminate with an aryl group when R¹ is mono-, di- or polyalkenyl, or mono-, di-, or polyalkynyl. Preferred R¹ groups include, but are not limited to, saturated fatty acid alkyl groups and mono-, di-, tri- and tetra-unsaturated alkenyl groups containing from 16 to 24 carbon atoms. The present invention provides a process as described in Example 1 for synthesizing multigram to kilogram quantities NPG and glucosamine amides of formula (I) according to the process described in Example 1.

As referred to herein, “alkyl” means an alkyl group up to and including 24 carbons. Common examples of such alkyl groups include methyl, ethyl, propyl, isopropyl, butyl, s-butyl, isobutyl, pentyl, neopentyl, hexyl, heptyl, isoheptyl, 2-ethylhexyl, cyclohexyl and octyl.

The term “aryl” as used herein is defined as a monovalent radical formed conceptually by removal of a hydrogen atom from a hydrocarbon that is structurally composed entirely of one or more benzene rings. Common examples of such hydrocarbons include benzene, biphenyl, terphenyl, naphthalene, phenyl naphthalene, and naphthylbenzene. Aryl is also meant to be an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), which is optionally mono-, di-, or trisubstituted with, e.g., halogen, lower alkyl, lower alkoxy, lower alkylthio, trifluoromethyl, lower acyloxy, aryl, heteroaryl, and hydroxy. By aryl is also meant heteroaryl groups where heteroaryl is defined as 5-, 6-, or 7-membered aromatic ring systems having at least one hetero atom selected from the group consisting of nitrogen, oxygen and sulfur. Examples of heteroaryl groups are pyridyl, pyrimidinyl, pyrrolyl, pyrazolyl, pyrazinyl, pyridazinyl, oxazolyl, furanyl, quinolinyl, isoquinolinyl, thiazolyl, and thienyl, which can optionally be substituted with, e.g., halogen, lower alkyl, lower alkoxy, lower alkylthio, trifluoromethyl, lower acyloxy, aryl, heteroaryl, and hydroxy.

As used herein, the term “alkaryl” denotes an aryl group which bears an alkyl group. One example of an alkaryl group is the 4-methylphenyl radical, C₇H₇, shown below:

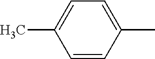

As used herein, the term “aralkyl” denotes an alkyl group which bears an aryl group; as used herein, the term “aralkyl” includes both substituted and unsubstituted groups. One such example is the benzyl group, i.e., the C₇H₇ radical shown below:

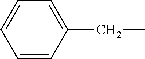

“Monoalkenyl” or “monoalkynyl” as used herein refers to the presence of a double or triple bond connecting the carbon atoms, respectively; dialkenyl or polyalkenyl refers to the presence of two or more double bond connected carbon atoms, polyalkynyl refers to two or more triple bond connected carbon atoms.

The term “agricultural composition” as used herein comprises one or more substances formulated for at least one agricultural application. Agricultural applications are understood to include, but not be limited to, yield improvement, pest control, disease control and resistance to abiotic environmental stress.

As used herein the term “biologically effective amount” refers to that amount of a substance required to produce the desired effect on plant growth and yield. Effective amounts of the composition will depend on several factors, including treatment method, plant species, propagating material type and environmental conditions.

The term “foliage” as used herein refers to the leaves of a plant.

Plant “growth” as used herein is defined by, but not limited to, measurements of seedling emergence, early growth, plant height, time to flowering, tillering (for grasses), days to maturity, vigor, biomass and yield.

As referred to in the present disclosure and claims, the term “propagating material” means a seed or regenerable plant part. The term “regenerable plant part” means a part of the plant other than a seed from which a whole plant may be grown or regenerated when the plant part is placed in agricultural or horticultural growing media such as moistened soil, peat moss, sand, vermiculite, perlite, rock wool, fiberglass, coconut husk fiber, tree fern fiber, and the like, or even a completely liquid medium such as water. Regenerable plant parts commonly include rhizomes, tubers, bulbs and corms of such geophytic plant species as potato, sweet potato, yam, onion, dahlia, tulip, narcissus, etc. Regenerable plant parts include plant parts that are divided (e.g., cut) to preserve their ability to grow into a new plant. Therefore regenerable plant parts include viable divisions of rhizomes, tubers, bulbs and corms which retain meristematic tissue, such as an eye. Regenerable plant parts can also include other plant parts such as cut or separated stems and leaves from which some species of plants can be grown using horticultural or agricultural growing media. As referred to in the present disclosure and claims, unless otherwise indicated, the term “seed” includes both unsprouted seeds and seeds in which the testa (seed coat) still surrounds part of the emerging shoot and root. Foliage as defined in the present application includes all aerial plant organs, that is, the leaves, stems, flowers and fruit.

The term “rhizosphere” as defined herein refers to the area of soil that immediately surrounds and is affected by the plant's roots.

As used herein, the term “treating” means applying a biologically effective amount of NPG, or a composition containing NPG, to a seed or other plant propagating material, plant foliage or plant rhizosphere; related terms such as “treatment” are defined analogously.

In one embodiment of the invention, the composition is applied as a seed treatment formulation. Such formulations typically contain from about 10⁻⁵ M to 10⁻¹² M of the composition. In a preferred embodiment, formulations contain from about 10⁻⁶ M to 10⁻¹⁰ M of a Formula I compound. The locus of the propagating materials can be treated with a Formula I compound by many different methods. All that is needed is for a biologically effective amount of a Formula I compound to be applied on or sufficiently close to the propagating material so that it can be absorbed by the propagating material. The Formula I compound can be applied by such methods as drenching the growing medium including a propagating material with a solution or dispersion of a Formula I compound, mixing a Formula I compound with growing medium and planting a propagating material in the treated growing medium (e.g., nursery box treatments), or various forms of propagating material treatments whereby a Formula I compound is applied to a propagating material before it is planted in a growing medium.

In these methods a Formula I compound will generally be used as a formulation or composition with an agriculturally suitable carrier comprising at least one of a liquid diluent, a solid diluent or a surfactant. A wide variety of formulations are suitable for this invention, the most suitable types of formulations depend upon the method of application. As is well known to those skilled in the art, the purpose of formulation is to provide a safe and convenient means of transporting, measuring and dispensing the agricultural agent and also to optimize its efficacy.

Depending on the method of application useful formulations include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films, and the like which can be water-dispersible (“wettable”) or water-soluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare. High-strength compositions are primarily used as intermediates for further formulation.

The formulations will typically contain effective amounts of active ingredient, diluent and surfactant within the following approximate ranges that add up to 100 percent by weight.

| | Weight Percent | | |
|---|---|---|---|
| | Active | | |
| | Ingredient | Diluent | Surfactant |
| Water-Dispersible and | 5-90 | 0-94 | 1-15 |
| Water-soluble Granules, | | | |
| Tablets and Powders. | | | |
| Suspensions, Emulsions, | 5-50 | 40-95 | 0-15 |
| Solutions (including | | | |
| Emulsifiable Concentrates) | | | |
| Dusts | 1-25 | 70-99 | 0-5 |
| Granules and Pellets | 0.01-99 | 5-99.99 | 0-15 |
| High Strength Compositions | 90-99 | 0-10 | 0-2 |

Typical solid diluents are described in Watkins et al., Handbook of Insecticide Dust Diluents and Carriers, 2nd Ed., Dorland Books, Caldwell, N.J. Typical liquid diluents are described in Marsden, Solvents Guide, 2nd Ed., Interscience, New York, 1950. McCutcheon's Emulsifiers and Detergents and McCutcheon's Functional Materials (North America and International Editions, 2001), The Manufactuing Confection Publ. Co., Glen Rock, N.J., as well as Sisely and Wood, Encyclopedia of Surface Active Agents, Chemical Publ. Co., Inc., New York, 1964, list surfactants and recommended uses. All formulations can contain minor amounts of additives to reduce foam, caking, corrosion, microbiological growth and the like, or thickeners to increase viscosity.

Surfactants include, for example, ethoxylated alcohols, ethoxylated alkylphenols, ethoxylated sorbitan fatty acid esters, ethoxylated amines, ethoxylated fatty acids, esters and oils, dialkyl sulfosuccinates, alkyl sulfates, alkylaryl sulfonates, organosilicones, N,N-dialkyltaurates, glycol esters, phosphate esters, lignin sulfonates, naphthalene sulfonate formaldehyde condensates, polycarboxylates, and block polymers including polyoxyethylene/polyoxypropylene block copolymers.

Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, propylene carbonate, dibasic esters, paraffins, alkylbenzenes, alkylnaphthalenes, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, and alcohols such as methanol, cyclohexanol, decanol, benzyl and tetrahydrofurfuryl alcohol.

Solutions, including emulsifiable concentrates, can be prepared by simply mixing the ingredients. Dusts and powders can be prepared by blending and, usually, grinding as in a hammer mill or fluid-energy mill. Suspensions are usually prepared by wet-milling; see, for example, U.S. Pat. No. 3,060,084. Granules and pellets can be prepared by spraying the active material upon preformed granular carriers or by agglomeration techniques. See Browning, “Agglomeration”, Chemical Engineering, Dec. 4, 1967, pp. 147-48, Perry's Chemical Engineer's Handbook, 4th Ed., McGraw-Hill, New York, 1963, pp. 8-57 and following, and PCT Publication WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp. 81-96; and Hance et al., Weed Control Handbook, 8th Ed., Blackwell Scientific Publications, Oxford, 1989.

The compositions used for treating propagating materials, or plants grown therefrom, according to this invention can also comprise (besides the Formula I component) an effective amount of one or more other biologically active compounds or agents. Suitable additional compounds or agents include, but are not limited to, insecticides, fungicides, nematocides, bactericides, acaricides, entomopathogenic bacteria, viruses or fungi, growth regulators such as rooting stimulants, chemosterilants, repellents, attractants, pheromones, feeding stimulants and other signal compounds including apocarotenoids, flavonoids, jasmonates and strigolactones (Akiyama, et al., in Nature, 435:824-827 (2005); Harrison, in Ann. Rev. Microbiol., 59:19-42 (2005); Besserer, et al., in PLoS Biol., 4(7):e226 (2006); WO2009049747). These compounds can also be formulated into mixtures or multi-component formulations.

Examples of such biologically active compounds or agents with which compounds of this invention can be mixed or formulated are: insecticides such as abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron; fungicides such as acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-dihydro-5-methyl-2-(methylthio)-5-phenyl-3-(phenylamino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin/fenominostrobin (SSF-126), metrafenone (AC 375839), myclobutanil, neo-asozin (ferric methanearsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin; nematocides such as aldicarb, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents including Bacillus thuringiensis (including ssp. aizawai and kurstaki), Bacillus thuringiensis delta-endotoxin, baculoviruses, and entomopathogenic bacteria, viruses and fungi. A general reference for these agricultural protectants is The Pesticide Manual, 12th Edition, C. D. S. Tomlin, Ed., British Crop Protection Council, Farnham, Surrey, U.K., 2000.

Preferred insecticides and acaricides for mixing or formulating with Formula I compounds include pyrethroids such as cypermethrin, cyhalothrin, cyfluthrin and beta-cyfluthrin, esfenvalerate, fenvalerate and tralomethrin; carbamates such as fenothicarb, methomyl, oxamyl and thiodicarb; neonicotinoids such as clothianidin, imidacloprid and thiacloprid; neuronal sodium channel blockers such as indoxacarb, insecticidal macrocyclic lactones such as spinosad, abamectin, avermectin and emamectin; γ-aminobutyric acid (GABA) antagonists such as endosulfan, ethiprole and fipronil; insecticidal ureas such as flufenoxuron and triflumuron; juvenile hormone mimics such as diofenolan and pyriproxyfen; pymetrozine; and amitraz. Preferred biological agents for mixing with compounds of this invention include Bacillus thuringiensis and Bacillus thuringiensis delta-endotoxin as well as naturally occurring and genetically modified viral insecticides including members of the family Baculoviridae as well as entomophagous fungi.

Preferred plant growth regulators for mixing or formulating with the Formula I compounds in compositions for treating stem cuttings are 1H-indole-3-acetic acid, 1H-indole-3-butanoic acid and 1-naphthaleneacetic acid and their agriculturally suitable salt, ester and amide derivatives, such as 1-napthaleneacetamide. Preferred fungicides for mixing with the Formula I compounds include fungicides useful as seed treatments such as thiram, maneb, mancozeb and captan.

For growing-medium drenches, the formulation needs to provide the Formula I compound, generally after dilution with water, in solution or as particles small enough to remain dispersed in the liquid. Water-dispersible or soluble powders, granules, tablets, emulsifiable concentrates, aqueous suspension concentrates and the like are formulations suitable for aqueous drenches of growing media. Drenches are most satisfactory for treating growing media that have relatively high porosity, such as light soils or artificial growing medium comprising porous materials such as peat moss, perlite, vermiculite and the like. The drench liquid comprising the Formula I compound can also be added to a liquid growing medium (i.e. hydroponics), which causes the Formula I compound to become part of the liquid growing medium. One skilled the art will appreciate that the amount of Formula I compound needed in the drench liquid for efficacy (i.e. biologically effective amount) will vary with several factors including, but not limited to, plant species, propagating material type and environmental conditions. The concentration of Formula I compound in the drench liquid is generally between about 10⁻⁵ M to 10⁻¹² M of the composition, more typically between about 10⁻⁶ M to 10⁻¹⁰ M. One skilled in the art can easily determine the biologically effective concentration necessary for the desired level of efficacy.

For treating a growing medium a Formula I compound can also be applied by mixing it as a dry powder or granule formulation with the growing medium. Because this method of application does not require first dispersing or dissolving in water, the dry powder or granule formulations need not be highly dispersible or soluble. While in a nursery box the entire body of growing medium may be treated, in an agricultural field only the soil in the vicinity of the propagating material is typically treated for environmental and cost reasons. To minimize application effort and expense, a formulation of Formula I compound is most efficiently applied concurrently with propagating material planting (e.g., seeding). For in-furrow application, the Formula I formulation (most conveniently a granule formulation) is applied directly behind the planter shoe. For T-band application, the Formula I formulation is applied in a band over the row behind the planter shoe and behind or usually in front of the press wheel. One skilled the art will appreciate that the amount of Formula I compound needed for efficacy (i.e. biologically effective amount) will vary with several factors including, but not limited to, plant species, propagating material type and environmental conditions. The concentration of Formula I compound in the growing medium locus is generally between about 10⁻⁵ M to 10⁻¹² M of the composition, more typically between about 10⁻⁶ M to 10⁻¹⁰ M. One skilled in the art can easily determine the biologically effective amount necessary for the desired level of efficacy.

A propagating material can be directly treated by soaking it in a solution or dispersion of a Formula I compound. Although this application method is useful for propagating materials of all types, treatment of large seeds (e.g., having a mean diameter of at least 3 mm) is more effective than treatment of small seeds for providing efficacy. Treatment of propagating materials such as tubers, bulbs, corms, rhizomes and stem and leaf cuttings also can provide effective treatment of the developing plant in addition to the propagating material. The formulations useful for growing-medium drenches are generally also useful for soaking treatments. The soaking medium comprises a nonphytotoxic liquid, generally water-based although it may contain nonphytotoxic amounts of other solvents such as methanol, ethanol, isopropanol, ethylene glycol, propylene glycol, propylene carbonate, benzyl alcohol, dibasic esters, acetone, methyl acetate, ethyl acetate, cyclohexanone, dimethylsulfoxide and N-methylpyrrolidone, which may be useful for enhancing solubility of the Formula I compound and penetration into the propagating material. A surfactant can facilitate wetting of the propagating material and penetration of the Formula I compound. One skilled the art will appreciate that the amount of Formula I compound needed in the soaking medium for efficacy (i.e. biologically effective amount) will vary with several factors including, but not limited to, plant species, propagating material type and environmental conditions. The concentration of Formula I compound in the soaking liquid is generally between about 10⁻⁵ M to 10⁻¹² M of the composition, more typically between about 10⁻⁶ M to 10⁻¹⁰ M. One skilled in the art can easily determine the biologically effective concentration necessary for the desired level of efficacy. The soaking time can vary from one minute to one day or even longer. Indeed, the propagating material can remain in the treatment liquid while it is germinating or sprouting (e.g., sprouting of rice seeds prior to direct seeding). As shoot and root emerge through the testa (seed coat), the shoot and root directly contact the solution comprising the Formula I compound. For treatment of sprouting seeds of large-seeded crops such as rice, treatment times of about 8 to 48 hours, e.g., about 24 hours, is typical. Shorter times are most useful for treating small seeds.

A propagating material can also be coated with a composition comprising a biologically effective amount of a Formula I compound. The coatings of the invention are capable of effecting a slow release of a Formula I compound by diffusion into the propagating material and surrounding medium. Coatings include dry dusts or powders adhering to the propagating material by action of a sticking agent such as methylcellulose or gum arabic. Coatings can also be prepared from suspension concentrates, water-dispersible powders or emulsions that are suspended in water, sprayed on the propagating material in a tumbling device and then dried. Formula I compounds that are dissolved in the solvent can be sprayed on the tumbling propagating material and the solvent then evaporated. Such compositions preferably include ingredients promoting adhesion of the coating to the propagating material. The compositions may also contain surfactants promoting wetting of the propagating material. Solvents used must not be phytotoxic to the propagating material; generally water is used, but other volatile solvents with low phytotoxicity such as methanol, ethanol, methyl acetate, ethyl acetate, acetone, etc. may be employed alone or in combination. Volatile solvents are those with a normal boiling point less than about 100° C. Drying must be conducted in a way not to injure the propagating material or induce premature germination or sprouting.

The thickness of coatings can vary from adhering dusts to thin films to pellet layers about 0.5 to 5 mm thick. Propagating material coatings of this invention can comprise more than one adhering layer, only one of which need comprise a Formula I compound. Generally pellets are most satisfactory for small seeds, because their ability to provide a biologically effective amount of a Formula I compound is not limited by the surface area of the seed, and pelleting small seeds also facilitates seed transfer and planting operations. Because of their larger size and surface area, large seeds and bulbs, tubers, corms and rhizomes and their viable cuttings are generally not pelleted, but instead coated with powders or thin films.

Propagating materials contacted with compounds of Formula I in accordance to this invention include seeds. Suitable seeds include seeds of wheat, durum wheat, barley, oat, rye, maize, sorghum, rice, wild rice, cotton, flax, sunflower, soybean, garden bean, lima bean, broad bean, garden pea, peanut, alfalfa, beet, garden lettuce, rapeseed, cole crop, turnip, leaf mustard, black mustard, tomato, potato, pepper, eggplant, tobacco, cucumber, muskmelon, watermelon, squash, carrot, zinnia, cosmos, chrysanthemum, sweet scabious, snapdragon, gerbera, babys-breath, statice, blazing star, lisianthus, yarrow, marigold, pansy, impatiens, petunia, geranium and coleus. Of note are seeds of cotton, maize, soybean and rice. Propagating materials contacted with compounds of Formula I in accordance to this invention also include rhizomes, tubers, bulbs or corms, or viable divisions thereof. Suitable rhizomes, tubers, bulbs and corms, or viable divisions thereof include those of potato, sweet potato, yam, garden onion, tulip, gladiolus, lily, narcissus, dahlia, iris, crocus, anemone, hyacinth, grape-hyacinth, freesia, ornamental onion, wood-sorrel, squill, cyclamen, glory-of-the-snow, striped squill, calla lily, gloxinia and tuberous begonia. Of note are rhizomes, tubers, bulbs and corms, or viable division thereof of potato, sweet potato, garden onion, tulip, daffodil, crocus and hyacinth. Propagating materials contacted with compounds of Formula I in accordance to this invention also include stems and leaf cuttings.

One embodiment of a propagating material contacted with a Formula I compound is a propagating material coated with a composition comprising a compound of Formula I and a film former or adhesive agent. Compositions of this invention which comprise a biologically effective amount of a compound of Formula I and a film former or adhesive agent, can further comprise an effective amount of at least one additional biologically active compound or agent. Of note are compositions comprising (in addition to the Formula I component and the film former or adhesive agent) an arthropodicides of the group consisting of pyrethroids, carbamates, neonicotinoids, neuronal sodium channel blockers, insecticidal macrocyclic lactones, γ-aminobutyric acid (GABA) antagonists, insecticidal ureas and juvenile hormone mimics. Also of note are compositions comprising (in addition to the Formula I component and the film former or adhesive agent) at least one additional biologically active compound or agent selected from the group consisting of abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron, aldicarb, oxamyl, fenamiphos, amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben, tebufenpyrad; and biological agents such as Bacillus thuringiensis (including ssp. aizawai and kurstaki), Bacillus thuringiensis delta-endotoxin, baculoviruses, and entomopathogenic bacteria, viruses and fungi. Also of note are compositions comprising (in addition to the Formula I component and the film former or adhesive agent) at least one additional biologically active compound or agent selected from fungicides of the group consisting of acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-dihydro-5-methyl-2-(methylthio)-5-phenyl-3-(phenylamino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin/fenominostrobin (SSF-126), metrafenone (AC 375839), myclobutanil, neo-asozin (ferric methanearsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin (especially compositions wherein the at least one additional biologically active compound or agent is selected from fungicides in the group consisting of thiram, maneb, mancozeb and captan).

Generally a propagating material coating of the invention comprises a compound of Formula I, a film former or sticking agent. The coating may further comprise formulation aids such as a dispersant, a surfactant, a carrier and optionally an antifoam and dye. One skilled the art will appreciate that the amount of Formula I compound needed for efficacy (i.e. biologically effective amount) will vary with several factors including, but not limited to, plant species, propagating material type and environmental conditions. The coating needs to not inhibit germination or sprouting of the propagating material.

The film former or adhesive agent component of the propagating material coating is composed preferably of an adhesive polymer that may be natural or synthetic and is without phytotoxic effect on the propagating material to be coated. The film former or sticking agent may be selected from polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxymethylcelluloses, hydroxy-propylcellulose, hydroxymethylpropylcelluloses, polyvinylpyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylimide polymers and copolymers, polyhydroxyethyl acrylate, methylacrylimide monomers, alginate, ethylcellulose, polychloroprene and syrups or mixtures thereof. Preferred film formers and adhesive agents include polymers and copolymers of vinyl acetate, poly-vinylpyrrolidone-vinyl acetate copolymer and water-soluble waxes. Particularly preferred are polyvinylpyrrolidone-vinyl acetate copolymers and water-soluble waxes. The above-identified polymers include those known in the art and for example some are identified as Agrimer® VA 6 and Licowax® KST. The amount of film former or sticking agent in the formulation is generally in the range of about 0.001 to 100% of the weight of the propagating material. For large seeds the amount of film former or sticking agent is typically in the range of about 0.05 to 5% of the seed weight; for small seeds the amount is typically in the range of about 1 to 100%, but can be greater than 100% of seed weight in pelleting. For other propagating materials the amount of film former or sticking agent is typically in the range of 0.001 to 2% of the propagating material weight.

Materials known as formulation aids may also be used in propagating material treatment coatings of the invention and are well known to those skilled in the art. Formulation aids assist in the production or process of propagating material treatment and include, but are not limited, to dispersants, surfactants, carriers, antifoams and dyes. Useful dispersants can include highly water-soluble anionic surfactants like Borresperse™ CA, Morwet® D425 and the like. Useful surfactants can include highly water-soluble nonionic surfactants like Pluronic® F108, Brij® 78 and the like. Useful carriers can include liquids like water and oils which are water-soluble such as alcohols. Useful carriers can also include fillers like woodflours, clays, activated carbon, diatomaceous earth, fine-grain inorganic solids, calcium carbonate and the like. Clays and inorganic solids which may be used include calcium bentonite, kaolin, china clay, talc, perlite, mica, vermiculite, silicas, quartz powder, montmorillonite and mixtures thereof. Antifoams can include water dispersible liquids comprising polyorganic siloxanes like Rhodorsil® 416. Dyes can include water dispersible liquid colorant compositions like Pro-Ized® Colorant Red. One skilled in the art will appreciate that this is a non-exhaustive list of formulation aids and that other recognized materials may be used depending on the propagating material to be coated and the compound of Formula I used in the coating. Suitable examples of formulation aids include those listed herein and those listed in McCutcheon's 2001, Volume 2: Functional Materials, published by MC Publishing Company. The amount of formulation aids used may vary, but generally the weight of the components will be in the range of about 0.001 to 10000% of the propagating material weight, with the percentages above 100% being mainly used for pelleting small seed. For nonpelleted seed generally the amount of formulating aids is about 0.01 to 45% of the seed weight and typically about 0.1 to 15% of the seed weight. For propagating materials other than seeds, the amount of formulation aids generally is about 0.001 to 10% of the propagating material weight.

Conventional means of applying seed coatings may be used to carry out the coating of the invention. Dusts or powders may be applied by tumbling the propagating material with a formulation comprising a Formula I compound and a sticking agent to cause the dust or powder to adhere to the propagating material and not fall off during packaging or transportation. Dusts or powders can also be applied by adding the dust or powder directly to the tumbling bed of propagating materials, followed by spraying a carrier liquid onto the seed and drying. Dusts and powders comprising a Formula I compound can also be applied by treating (e.g., dipping) at least a portion of the propagating material with a solvent such as water, optionally comprising a sticking agent, and dipping the treated portion into a supply of the dry dust or powder. This method can be particularly useful for coating stem cuttings. Propagating materials can also be dipped into compositions comprising Formula I formulations of wetted powders, solutions, suspoemulsions, emulfiable concentrates and emulsions in water, and then dried or directly planted in the growing medium. Propagating materials such as bulbs, tubers, corms and rhizomes typically need only a single coating layer to provide a biologically effective amount of a Formula I compound.

Propagating materials may also be coated by spraying a suspension concentrate directly into a tumbling bed of propagating materials and then drying the propagating materials. Alternatively, other formulation types like wetted powders, solutions, suspoemulsions, emulsifiable concentrates and emulsions in water may be sprayed on the propagating materials. This process is particularly useful for applying film coatings to seeds. Various coating machines and processes are available to one skilled in the art. Suitable processes include those listed in P. Kosters et al., Seed Treatment: Progress and Prospects, 1994 BCPC Monograph No. 57 and the references listed therein. Three well-known techniques include the use of drum coaters, fluidized bed techniques and spouted beds. Propagating materials such as seeds may be presized prior to coating. After coating the propagating materials are dried and then optionally sized by transfer to a sizing machine. These machines are known in the art for example, as a typical machine used when sizing corn (maize) seed in the industry.

For coating seed, the seed and coating material are mixed in any variety of conventional seed coating apparatus. The rate of rolling and coating application depends upon the seed. For large oblong seeds such as those of cotton, a satisfactory seed coating apparatus comprises a rotating type pan with lifting vanes turned at sufficient rpm to maintain a rolling action of the seed, facilitating uniform coverage. For seed coating formulations applied as liquids, the seed coating must be applied over sufficient time to allow drying to minimize clumping of the seed. Using forced air or heated forced air can facilitate an increased rate of application. One skilled in the art will also recognize that this process may be a batch or continuous process. As the name implies, a continuous process allows the seeds to flow continuously throughout the product run. New seeds enter the pan in a steady stream to replace coated seeds exiting the pan.

The seed coating process of the present invention is not limited to thin film coating and may also include seed pelleting. The pelleting process typically increases the seed weight from 2 to 100 times and can be used to also improve the shape of the seed for use in mechanical seeders. Pelleting compositions generally contain a solid diluent, which is typically an insoluble particulate material, such as clay, ground limestone, powdered silica, etc., to provide bulk in addition to a binder such as an artificial polymer (e.g., polyvinyl alcohol, hydrolyzed polyvinyl acetates, polyvinyl methyl ether, polyvinyl methyl ether-maleic anhydride copolymer, and polyvinylpyrrolidinone) or natural polymer (e.g., alginates, karaya gum, jaguar gum, tragacanth gum, polysaccharide gum, mucilage). After sufficient layers have been built up, the coat is dried and the pellets graded. A method for producing pellets is described in Agrow, The Seed Treatment Market, Chapter 3, PJB Publications Ltd., 1994.

Seed varieties and seeds with specific transgenic traits may be tested to determine which seed treatment options and application rates may complement such varieties and transgenic traits in order to enhance yield. Further, the good root establishment and early emergence that results from the proper use of the compound of formula I seed treatment may result in more efficient nitrogen use, a better ability to withstand drought and an overall increase in yield potential of a variety or varieties containing a certain trait when combined with a seed treatment.

In another embodiment of the invention, the composition is applied as a foliar formulation. Such formulations will generally include at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake. Effective foliar formulations will typically contain from about 10⁻⁵ M to 10⁻¹² M of the composition. In a preferred embodiment, formulations contain from about 10⁻⁶ M to 10⁻¹⁰ M of the compound of formula I.

In another embodiment of the invention, the composition is applied to soil either prior to or following planting of plant propagating materials. Compositions can be applied as a soil drench of a liquid formulation, a granular formulation to the soil, a nursery box treatment or a dip of transplants. Of note is a composition of the present invention in the form of a soil drench liquid formulation. Of further note is this method wherein the environment is soil and the composition is applied to the soil as a soil drench formulation. Other methods of contact include application of a compound or a composition of the invention by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the invention. Effective soil formulations will typically contain from about 10⁻⁵ M to 10⁻¹² M of the composition. In a preferred embodiment, formulations contain from about 10⁻⁶ M to 10⁻¹⁰ M of the compound of formula I.

The method of this invention is applicable to virtually all plant species. Seeds that can be treated include, for example, wheat (Triticum aestivum L.), durum wheat (Triticum durum Desf.), barley (Hordeum vulgare L.), oat (Avena sativa L.), rye (Secale cereale L.), maize (Zea mays L.), sorghum (Sorghum vulgare Pers.), rice (Oryza sativa L.), wild rice (Zizania aquatica L.), millet (Eleusine coracana, Panicum miliaceum), cotton (Gossypium barbadense L. and G. hirsutum L.), flax (Linum usitatissimum L.), sunflower (Helianthus annuus L.), soybean (Glycine max Merr.), garden bean (Phaseolus vulgaris L.), lima bean (Phaseolus limensis Macf.), broad bean (Vicia faba L.), garden pea (Pisum sativum L.), peanut (Arachis hypogaea L.), alfalfa (Medicago sativa L.), beet (Beta vulgaris L.), garden lettuce (Lactuca sativa L.), rapeseed (Brassica rapa L. and B. napus L.), cole crops such as cabbage, cauliflower and broccoli (Brassica oleracea L.), turnip (Brassica rapa L.), leaf (oriental) mustard (Brassica juncea Coss.), black mustard (Brassica nigra Koch), tomato (Lycopersicon esculentum Mill.), potato (Solanum tuberosum L.), pepper (Capsicum frutescens L.), eggplant (Solanum melongena L.), tobacco (Nicotiana tabacum), cucumber (Cucumis sativus L.), muskmelon (Cucumis melo L.), watermelon (Citrullus vulgaris Schrad.), squash (Curcurbita pepo L., C. moschata Duchesne. and C. maxima Duchesne.), carrot (Daucus carota L.), zinnia (Zinnia elegans Jacq.), cosmos (e.g., Cosmos bipinnatus Cay.), chrysanthemum (Chrysanthemum spp.), sweet scabious (Scabiosa atropurpurea L.), snapdragon (Antirrhinum majus L.), gerbera (Gerbera jamesonii Bolus), babys-breath (Gypsophila paniculata L., G. repens L. and G. elegans Bieb.), statice (e.g., Limonium sinuatum Mill., L. sinense Kuntze.), blazing star (e.g., Liatris spicata Willd., L. pycnostachya Michx., L. scariosa Willd.), lisianthus (e.g., Eustoma grandiflorum (Raf.) Shinn), yarrow (e.g., Achillea filipendulina Lam., A. millefolium L.), marigold (e.g., Tagetes patula L., T. erecta L.), pansy (e.g., Viola cornuta L., V. tricolor L.), impatiens (e.g., Impatiens balsamina L.) petunia (Petunia spp.), geranium (Geranium spp.) and coleus (e.g., Solenostemon scutellarioides (L.) Codd). Not only seeds, but also rhizomes, tubers, bulbs or corms, including viable cuttings thereof, can be treated according to the invention from, for example, potato (Solanum tuberosum L.), sweet potato (Ipomoea batatas L.), yam (Dioscorea cayenensis Lam. and D. rotundata Poir.), garden onion (e.g., Allium cepa L.), tulip (Tulipa spp.), gladiolus (Gladiolus spp.), lily (Lilium spp.), narcissus (Narcissus spp.), dahlia (e.g., Dahlia pinnata Cay.), iris (Iris germanica L. and other species), crocus (Crocus spp.), anemone (Anemone spp.), hyacinth (Hyacinth spp.), grape-hyacinth (Muscari spp.), freesia (e.g., Freesia refracta Klatt., F. armstrongii W. Wats), ornamental onion (Allium spp.), wood-sorrel (Oxalis spp.), squill (Scilla peruviana L. and other species), cyclamen (Cyclamen persicum Mill. and other species), glory-of-the-snow (Chionodoxa luciliae Boiss. and other species), striped squill (Puschkinia scilloides Adams), calla lily (Zantedeschia aethiopica Spreng., Z. elliottiana Engler and other species), gloxinia (Sinnigia speciosa Benth. & Hook.) and tuberous begonia (Begonia tuberhybrida Voss.). Stem cuttings can be treated according to this invention include those from such plants as sugarcane (Saccharum officinarum L.), carnation (Dianthus caryophyllus L.), florists chrysanthemum (Chrysanthemum mortifolium Ramat.), begonia (Begonia spp.), geranium (Geranium spp.), coleus (e.g., Solenostemon scutellarioides (L.) Codd) and poinsettia (Euphorbia pulcherrima Willd.). Leaf cuttings which can be treated according to this invention include those from begonia (Begonia spp.), african-violet (e.g., Saintpaulia ionantha Wendl.) and sedum (Sedum spp.). The above recited cereal, vegetable, ornamental (including flower) and fruit crops are illustrative, and should not be considered limiting in any way. For reasons of economic importance, preferred embodiments of this invention include wheat, rice, maize, barley, sorghum, oats, rye, millet, soybeans, peanuts, beans, rapeseed, canola, sunflower, sugar cane, potatoes, sweet potatoes, cassava, sugar beets, tomatoes, plantains and bananas, and alfalfa.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### Example 1

### Synthesis of N-palmitoleyl-D-glucosamine (NPG)

Unless specified, all the reagents were purchased from Aldrich Chemical Co (St. Louis, Mo.). Thin layer chromatography was performed on pre-coated plates of Silica Gel 60 F₂₅₄ (EM Science) and the spots were visualized with a spray containing 5% sulfuric acid in ethanol, followed by heating. Column chromatography was done on silica gel 60 (230-400 mesh, EM Science). ¹H NMR spectra were recorded at 500 MHz. The hydrogen chemical shifts in organic solvents are expressed relative to deuterated methylenechloride, with a reference chemical shift of 5.36 ppm. For solutions of compounds in deuterium oxide or deuterated methanol, the hydrogen chemical shift values are expressed relative to the HOD signal (4.75 ppm at 296° K).

### Synthesis of 2-deoxy-1,3,4,6-tetra-O-acetyl-2-phthalimido-D-glucopyranose

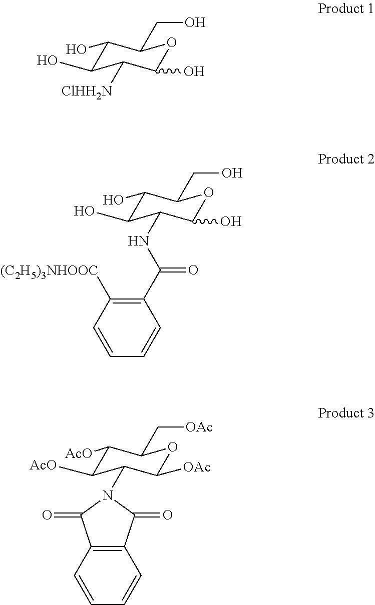

D-Glucosamine hydrochloride (Product 1, 1.0 kg) was suspended in methanol (5.0 L) and vigorously stirred. NaOH (184.8 g) was dissolved in minimum deionized water and added to the D-glucosamine/methanol suspension. The suspension was stirred for 15 min and the insoluble material (sodium chloride) was filtered off by vacuum filtration. The theoretical amount of NaCl formed should be about 270 g.

To the filtrate, phthalic anhydride (342 g) was added and the solution was stirred until most of the solid dissolved (about 30 min). This was then followed by the addition of triethylamine (468 g) and stirred for 10 to 15 min. To the resulting clear solution, another portion of phthalic anhydride (342 g) was added and the mixture was allowed to stir overnight at room temperature. Product usually began to precipitate out after two hours.

The precipitated product was filtered and the residue was washed with minimum ice cold methanol so as to remove the yellow color from the product. The residue was then washed three times with acetonitrile, with enough solvent added to the filter to completely immerse the solid, and dried at room temperature under high vacuum. The weight of the white solid, Product 2, was 954 g. ¹H-NMR (D₂O): 7.74-7.56 (phthalimido hydrogens), 5.42 (H-1α), 4.94 (H-1β), 4.17 and 4.01 (H-6), 3.27 (CH₂ of N-ethyl group), 1.35 (CH₃ of N-ethyl group).

The Product 2 from above (1.01 kg, made from two batches) was placed in a 10 liter 3 neck round bottom flask set up with an overhead electric stirrer, an N₂ inlet and an addition funnel. Acetic anhydride (3 L) and N,N-dimethylaminopyridine (1.0 g) were added to the flask and stirred vigorously. Pyridine (2.8 L) was added slowly and the reaction mixture was stirred for two days at room temperature. The reaction mixture was quenched with ice-water (4 L) and the product was extracted with methylenechloride. The organic layer was repeatedly washed with aqueous hydrochloric acid solution, and then with saturated sodium bicarbonate solution. The organic layer was dried over anhydrous magnesium sulfate, filtered, and concentrated to dryness. The product was recrystallized from hot ethanol. Weight of the recrystallized Product 3 was 701 g. ¹H-NMR (CD₂Cl₂) δ: 7.91-7.80 (phthalimido hydrogens), 6.62 (H-1), 5.59 (H-3), 5.21 (H-4), 4.47 (H-2), 4.36 and 4.16 (H-6), 4.06 (H-5), 2.12, 2.06, 2.02, 1.88 (acetyl methyl groups). Thus, the above NMR chemical shift data verified the structure of product 3,2-deoxy-1,3,4,6-tetra-O-acetyl-2-phthalimido-D-glucopyranose.

### Preparation of Product of Formula 7 (NPG)

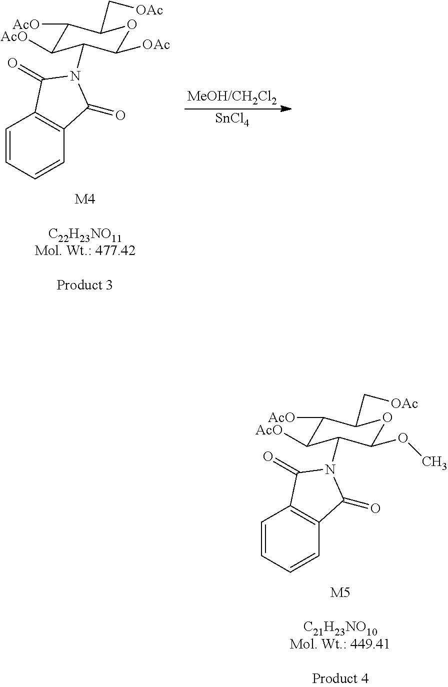 [SMILES: CC(=O)OCC1OC(OC(C)=O)C(N2C(=O)c3ccccc3C2=O)C(OC(C)=O)C1OC(C)=O ; "SnCl4" ; COC1OC(COC(C)=O)C(OC(C)=O)C(OC(C)=O)C1N1C(=O)c2ccccc2C1=O]

To ensure that the starting glycoside was free of EtOH traces, Product 3 (60.0 g; 126 mmol) was dissolved in toluene and evaporated. It was then dissolved in anhydrous CH₂Cl₂ (500 ml) containing MeOH (6.5 g; 202 mmol; 1.6 eq.). Tin tetrachloride (SnCl₄; 18.4 g; 70.5 mmol; 0.56 eq.) was diluted with CH₂Cl₂ (25 ml) and added drop-wise. The reaction mixture was poured over ice water and shaken well. This was repeated once more and then the organic layer was washed twice with aqueous saturated NaHCO₃, dried with MgSO₄, filtered, and concentrated. The crude product was recrystallized from hot EtOH, giving crystals of Product 4 (43.1 g). The crude yield of 49.8 g of Product 4 was 88% of the theoretical yield, calculated to be 56.6 g, while the recrystallized Product 4 yield of 43.1 g was 76%.

¹H-NMR (CD₂Cl₂) δ: 7.86-7.74 (phthalimido hydrogens), 5.78 (H-3), 5.31 (H-1), 5.18 (H-4), 4.31 (H-2), 4.34 & 4.20 (H-6), 3.88 (H-5), 2.20, 2.03, 1.86 (methyls of acetyl groups). Thus the NMR spectrum verified the structure of Product 4, as shown above.

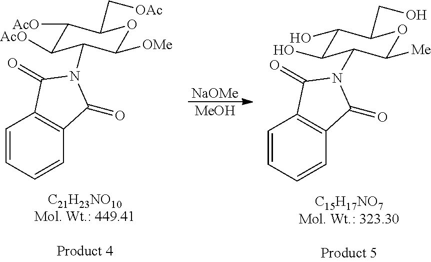 [SMILES: "MeOH" ; COC1OC(COC(C)=O)C(OC(C)=O)C(OC(C)=O)C1N1C(=O)c2ccccc2C1=O ; CC1OC(CO)C(O)C(O)C1N1C(=O)c2ccccc2C1=O ; "NaOMe"]

Product 4 (141.0 g; 314 mmol) was suspended in MeOH (1000 ml), and NaOMe (0.5 M, 10 ml) was added. The methyl glycoside Product 4 did not readily dissolve in MeOH. The solution was tested to ensure basicity. The reaction was stirred overnight. The solution became clear. Examination of the reaction mixture by TLC (EtOAc-Hexane-EtOH=10:20:1) indicated the disappearance of the starting material and the formation of a polar product (near the origin). The solution was neutralized with sulfonic acid resin, filtered, and concentrated to dryness. Weight of the residue, called Product 5, was 105.3 g, which probably includes some methanol.

The crude yield of 105.3 g of Product 5 was essentially equal to the theoretical yield, calculated to be 101.3 g. ¹H-NMR (CD₃OD) δ: 7.85-7.80 (phthalimido hydrogens), 5.07 (H-1), 4.21 (H-2), 3.94 (H-3), 3.92 & 3.74 (H-6), 3.40 (H-5), 3.40 (OCH₃), 3.38 (H-4). Thus the NMR spectrum verified the structure of Product 5, as shown above.

### Synthesis of Product 6

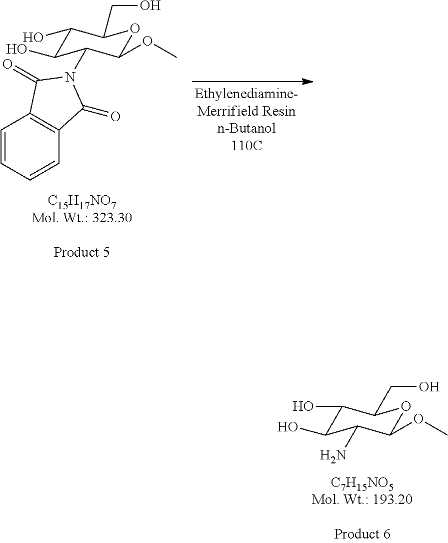 [SMILES: COC1OC(CO)C(O)C(O)C1N1C(=O)c2ccccc2C1=O ; COC1OC(CO)C(O)C(O)C1N]

The trihydroxy methylglycoside (Product 5, 5.00 g), ethylenediamine modified Merrifield resin (25.21 g) and n-butanol (100 mL) were combined in a round bottom flask that was fitted with a reflux condenser and placed under a dry N₂ atmosphere. The mixture was stirred and heated to 110° C. using an oil bath. The reaction was left to stir at this temperature overnight. At this time, the reaction progress was checked by TLC using a 5:1:5 ethyl acetate/hexanes/ethanol eluant, which showed the presence of small amount of the starting material. To ensure total conversion, additional portions of ethylenediamine Merrifield resin (10.00 g) and n-butanol (20 mL) were added and the reaction was allowed to stir at reflux temperature for three more hours. Following this, the reaction mixture was filtered warm and the resin that deposited in the filter funnel was washed with methanol (50 mL, 3 times). The combined filtrate was reduced to dryness, giving Product 6 (4.00 g), the structure of which was confirmed by proton NMR.

### Reaction B: Addition of cis-(C16:1)-COOH Fatty Acid

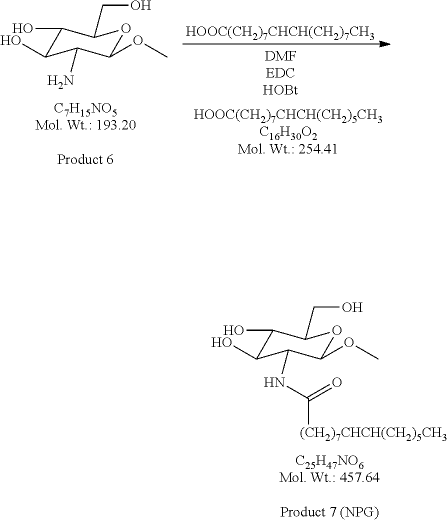

Product 6=2.99 g, 15.47 mmol

Cis-CH₃(CH₂)₅CHCH(CH₂)₇COOH (FW 254.41): 1.1 eq, 17.02 mmol, 4.33 g

EDC (FW 191.62): 1.2 eq, 18.56 mmol, 3.56 g

HOBt-H₂O (FW 153.15): 1.1 eq, 17.02 mmol, 2.61 g

DMF: 55 mL

Temperature: 20° C.

Reaction Time: overnight

Theoretical Yield: 7.08 g

Purified Yield: 5.78 g (81.6%)

### Procedure:

In the drybox, the cis-CH₃(CH₂)₅CHCH(CH₂)₇COOH fatty acid (1.1 eq, 17.02 mmol, 4.33 g) and DMF (30 mL) were combined in an vial and the EDC (1.2 eq, 18.56 mmol, 3.56 g) and HOBt-H₂O (1.1 eq, 17.02 mmol, 2.61 g) were added to this mixture. Product 6 (2.99 g, 15.47 mmol) was dissolved in DMF and added slowly (drop-wise) to the clear colorless fatty acid/EDC/HOBt solution, resulting in a clear light yellow reaction mixture. Approximately 60-75 mL DMF was used in the reaction. The reaction was stirred for 6 h and the progress was checked by TLC using a 4:2:1 ethyl acetate/ethanol/water as the eluant, which showed that all of the starting material was consumed. The reaction was left to stir overnight and analyzed by 1H NMR, which showed that the reaction was complete. The reaction mixture was concentrated to obtain a waxy material.

### Purification of Product 7 (NPG)—Methyl 2-deoxy-2-N-(hexadece-9,10-cis-eneoyl)-β-D-glucopyranoside

The crude product was dissolved in CH₂Cl₂ (25 mL). This solution was stirred while during slow addition of water (25 mL). A white precipitate formed and this solid was filtered.

The filtrates were transferred back to the round bottom flask and all of the equipment used for filtration was rinsed clean with CH₂Cl₂. Additional water was added to this mixture and vigorously stirred. This resulted in an emulsion. The mixture was left standing and the water layer that separated at the top was decanted off. The remaining mixture was concentrated to remove the CH₂Cl₂, resulting in a thick white foamy product. This was re-suspended in CH₂Cl₂ (˜200 mL) and the mixture was stirred. This resulted in a clear yellow CH₂Cl₂ solution and a white emulsion. The entire mixture was transferred to a separatory funnel and the CH₂Cl₂ layer was collected. Additional CH₂Cl₂ was added (100 mL) to the separatory funnel and the mixture was shaken in an attempt to extract the product form the emulsion. The CH₂Cl₂ layer (clear, but slightly white) was collected and the extraction repeated two additional times. The CH₂Cl₂ extractions were combined and concentrated to dryness. This had essentially the desired product contaminated with small amounts of HOBt.

This material was re-suspended CH₂Cl₂ (400 mL), resulting in a majority of the solid dissolving with some fluffy white material remaining suspended. This mixture was stirred and anhydrous MgSO₄ was added to remove any residual water; however, the fluffy suspended solids remained (this turned out to be the desired product). This mixture was filtered and the filter cake was suspended in a mixture of acetonitrile-methanol (1:2). This mixture was filtered and the filtrate was reduced to dryness, giving the desired Product 7 (NPG), the structure of which was confirmed by proton NMR.

### Example 2

### Effect of NPG on Plant Emergence, Flowering and Yield of Potatoes Grown Under Field Conditions in 2010

### Materials and Methods

A potato field trial was conducted to evaluate the effects of NPG on plant emergence, flowering and yield in Shepody potatoes (Solanum tuberosum) planted near Thorndale, Ontario, Canada, in 2010. Seed treatments included an Untreated Control, three concentrations of NPG and three concentrations of an LCO derived from Bradyrhizobium japonicum to serve as a positive control. The LCO was provided by Dr. Don Smith (McGill University, Montreal, Canada) using the basic method described in Soulemanov, A., et al., in Microbiol. Res., 157: 25-28 (2005). Aqueous solutions of NPG and LCO were applied at 10⁻⁶M, 10⁻⁷M and 10⁻⁸M to seed pieces using a spray nozzle and left to soak on a plastic sheet for 20 minutes. The seed pieces were beginning to sprout when planted on June 8^{th} in a loam composed of 41% sand, 39% silt and 20% clay and 4.5% organic matter. The soil had a pH of 6.9 and cationic exchange capacity of 14.7.

Potatoes were planted at a rate of 33,300 seed pieces/ha to a depth of 20 cm and hilled using a tractor mounted potato hiller. Weeds were controlled using three L/ha of 40.6 wt % linuron. Insects were controlled using 250 ml/ha 18.4 wt % chlorantraniprole, and disease was controlled using a tank mix of 1.6 kg/ha 75 wt % mancozeb and 225 g/ha 60 wt % cymoxanil fungicides. Before harvest, plants were sprayed twice (seven days apart) with 39.5 wt % diquat dibromide herbicide at a rate of 2 L/ha. All of the products used are industry standards and representative of what is used in commercial production.

The trial was conducted using a randomized complete block design with a plot size of 2 m by 8 m with a 100 cm row spacing, 30 cm plant spacing and four replications Due to wet weather, the soil was damp and cloddy during the planting and hilling process.

### Results

Percent emergence was observed at 15, 17, 21, and 29 days after planting (DAP) (Table 1). All three concentrations of NPG significantly increased emergence rates versus the Untreated Control at 21 and 29 DAP. The highest concentration of NPG (10⁻⁶ M) also significantly increased emergence rates versus the Untreated Control at 15 and 17 DAP. A non-statistically significant improvement was observed for the two lower NPG concentrations (10⁻⁷ M and 10⁻⁸M) at 15 and 17 DAP. NPG and LCO treatments provided similar results, with 10⁻⁶ M NPG and 10⁻⁸ M LCO exhibiting the greatest overall efficacy.

TABLE 1
Effect of NPG on Time to Potato Emergence (%)

| Treatment | 15 DAP | 17 DAP | 21 DAP | 29 DAP |
|---|---|---|---|---|
| Untreated | 5a | 17a | 28a | 31a |
| 10⁻⁶M NPG | 16b | 41b | 68c | 72c |
| 10⁻⁷M NPG | 9ab | 30ab | 48b | 52b |
| 10⁻⁸M NPG | 13ab | 30ab | 45b | 55b |
| 10⁻⁶M LCO | 12ab | 36ab | 55b | 60bc |
| 10⁻⁷M LCO | 16b | 30ab | 54b | 57bc |
| 10⁻⁸M LCO | 14ab | 39b | 71c | 77d |

Treatments followed by the same letter in a column are not significantly different when compared using Tukey's LSD test.

Percent flowering was observed 42 and 50 days following NPG application (Table 2). A non-statistically significant increase in flowering percentage at 50 DAP was observed for all NPG and LCO treatments compared to the Untreated Control.

TABLE 2
Effect of NPG on time to Potato Flowering (%)

| Treatment | 42 DAP | 50 DAP |
|---|---|---|
| Untreated | 19a | 31a |
| 10⁻⁶M NPG | 19a | 53ab |
| 10⁻⁷M NPG | 20a | 47ab |
| 10⁻⁸M NPG | 11a | 54ab |
| 10⁻⁶M LCO | 13a | 40ab |
| 10⁻⁷M LCO | 17a | 63ab |
| 10⁻⁸M LCO | 23a | 35a |

Treatments followed by the same letter in a column are not significantly different when compared using Tukey's LSD test.

Marketable potato yield was determined by harvesting one entire 8 m row of the plot (Table 3). The Untreated Control row with the highest emergence was harvested. Yields were not corrected for differences in emergence rates. The 10⁻⁶ M and 10⁻⁷ M NPG treatments and the 10⁻⁸ M LCO treatment provided a statistically significant increase in marketable fresh weight yield compared to the Untreated Control. The remaining experimental treatments exhibited a non-statistically significant yield increase versus the Untreated Control.

TABLE 3
Effect of NPG on Marketable Potato Yield for One Row of Plot

| Treatment | Kg/Plot |
|---|---|
| Untreated | 7.42a |
| 10⁻⁶M NPG | 17.92b |
| 10⁻⁷M NPG | 16.05b |
| 10⁻⁸M NPG | 13.80ab |
| 10⁻⁶M LCO | 15.60ab |
| 10⁻⁷M LCO | 15.11ab |
| 10⁻⁸M LCO | 17.47b |

Treatments followed by the same letter in a column are not significantly different when compared using Tukey's LSD test.

### Example 3

### Effect of NPG on Plant Emergence, Flowering, Vigor and Biomass of Potatoes Grown Under Field Conditions in 2011

### Materials and Methods

A potato field trial was conducted to evaluate the effects of NPG on plant emergence, flowering, vigor and biomass in Superior potatoes planted near Breslau, Ontario, Canada, in 2011. Seed treatments included an Untreated Control, NPG applied as a seed treatment, and NPG applied as a seed treatment followed by two foliar applications. Aqueous solutions of NPG were applied at a 10⁻⁷ M concentration to seed pieces using a spray nozzle and left to soak on a plastic sheet for 20 min.

Foliar NPG applications were performed 36 and 45 days after planting. Plants were sprayed using a four-nozzle hollow-cone boom containing ceramic disks and CO₂ propellant at a speed of 4.5 km/h and 40 psi. The 36 day application utilized a 2.0 L mix size and spray rate of 200 L/ha water volume. The 45 day application utilized a 3.0 L mix size and spray rate of 300 L/ha water volume. Seeds pieces designated for use as the Untreated Control also received the fungicide maintenance treatment.

The seed pieces were beginning to sprout when planted on June 8^{th} in a loam composed of 34% sand, 48% silt and 18% clay and 2.9% organic matter. The soil had a pH of 7.4 and cationic exchange capacity of 19.3.

Potatoes were planted at a rate of 25,000 seed pieces/ha to a depth of 20 cm and hilled using a tractor mounted potato hiller. Weeds were controlled using 3 L/ha of 40.6 wt % linuron. Insects were controlled using 250 ml/ha 18.4 wt % chlorantraniliprole and 80 g/ha 70 wt % acetamiprid insecticides. Disease was controlled using a tank mix of 1.6 kg/ha 75 wt % mancozeb M and 225 g/ha 60 wt % cymoxanil fungicides. Before harvest, plants were sprayed twice (seven days apart) with 39.5 wt % diquat dibromide herbicide at a rate of 2 L/ha. All of the products are industry standards and representative of what is used in commercial production.

The trial was conducted using a randomized complete block design with a plot size of 2 m by 8 m with a 100 cm row spacing, 30 cm plant spacing and four replications. Due to wet weather, the soil was damp and cloddy during the planting and hilling process.

### Results

Percent emergence was observed at 20 and 28 DAP (Table 4). No significant differences were observed with either NPG treatment compared to the Untreated Control.

TABLE 4
Effect of NPG on Time to Potato Emergence (%)

| Treatment | 20 DAP | 28 DAP |
|---|---|---|
| Untreated | 37a | 40a |
| 10⁻⁷M NPG | 30a | 39a |
| 10⁻⁷M NPG + Foliar | 36a | 38a |

Treatments followed by the same letter in a column are not significantly different when compared using Tukey's LSD test.

Time to flowering was observed 40 days after NPG application (Table 5). A significant increase in flowering numbers was observed with the NPG+Foliar treatment compared to the Untreated Control.

TABLE 5
Effect of NPG on Time to Potato Flowering (%)

| Treatment | 40 DAP |
|---|---|
| Untreated | 0.5a |
| 10⁻⁷M NPG | 0.0a |
| 10⁻⁷M NPG + Foliar | 22b |

Treatments followed by the same letter in a column are not significantly different when compared using Tukey's LSD test.

Crop vigor was observed at 20, 28, 40, and 48 DAP (Table 6). Treatments were compared on a “Vigor Scale” of 1 to 5 as follows:

1=Visually inferior to untreated check
2=Slightly worse than untreated check
3=Same as untreated check
4=Slightly better than untreated check
5=Visually superior to untreated check

Both NPG treatments exhibited a statistically significant increase in crop vigor versus the Untreated Control at 49 DAP. No significant differences between treatments were observed at 20, 28 and 40 DAP; however, the NPG+Foliar treatment showed a direction improvement at these time points.

TABLE 6
Effect of NPG on Crop Vigor (1-5 Scale)

| Treatment | 20 DAP | 28 DAP | 40 DAP | 49 DAP |
|---|---|---|---|---|
| Untreated | 3.0a | 3.0a | 3.0a | 3.0a |
| 10⁻⁷M NPG | 2.9a | 2.9a | 3.1a | 3.5b |
| 10⁻⁷M NPG + Foliar | 4.6b | 3.9a | 3.6a | 4.1c |

Treatments followed by the same letter in a column are not significantly different when compared using Tukey's LSD test.

Potato Plant Biomass was determined at 117 DAP by harvesting five plants above ground level from each plot (Table 7). A non-statistically significant biomass increase was observed for both NPG treatments versus the Untreated Control.

TABLE 7
Effect of NPG on Plant Biomass

| Treatment | kg/5 Plants |
|---|---|
| Untreated | 2.1a |
| 10⁻⁷M NPG | 2.35a |
| 10⁻⁷M NPG + Foliar | 2.56a |

Treatments followed by the same letter in a column are not significantly different when compared using Tukey's LSD test.

### Example 4

### Effect of NPG on Plant Emergence, Height and Yield of Spring Barley Grown Under Field Conditions in 2010

### Materials and Methods

A field trial was conducted to evaluate the effects of NPG on plant emergence, height, and yield in Spring barley (Hordeum vulgare) planted near Thorndale, Ontario, Canada, in 2010. Seed treatments included an Untreated Control, three concentrations of NPG, and three concentrations of a natural LCO. The natural LCO was provided by Dr. Don Smith (McGill University, Montreal, Canada) and prepared as described in Example 2. Aqueous solutions of NPG and LCO were applied at 10⁻⁶ M, 10⁻⁷ M and 10⁻⁸M to barley seed using a spray nozzle and left to soak on a plastic sheet for 20 minutes. The seeds were beginning to emerge when they were planted on June 8^{th} in a loam composed of 41% sand, 39% silt and 20% clay and 4.5% organic matter. The soil had a pH of 6.9 and cationic exchange capacity of 14.7.

Barley was planted at a rate of 100 kg seed/ha to a depth of 2.5 cm. Weeds were controlled using 770 mL/ha 8.79 wt % fenoxprop-P-ethyl, and a mixture of 33.33 wt % thifensulfuron methyl and 16.67 wt % tribenuron methyl at a rate of 30 g/ha. Disease was controlled with 23.6 wt % pyraclostrobin at a rate of 0.4 L/ha. All of the products are industry standards and representative of what is used in commercial production.

The trial was conducted using a randomized complete block design with a plot size of 2 m by 8 m with a 17.8 cm row spacing, 3.3 cm plant spacing and four replications

### Results

Percent emergence was observed at 7, 15, and 43 DAP (Table 8). NPG and LCO treatments significantly increased emergence rates versus the Untreated Control at all three time points. NPG and LCO treatments exhibited comparable efficacy.

TABLE 8
Effect of NPG on Time to Barley Emergence (%)

| Treatment | 7 DAP | 15 DAP | 43 DAP |
|---|---|---|---|
| Untreated | 67b | 74c | 1b |
| 10⁻⁶M NPG | 85a | 88ab | 19a |
| 10⁻⁷M NPG | 86a | 91a | 30a |
| 10⁻⁸M NPG | 83a | 86ab | 25a |
| 10⁻⁶M LCO | 89a | 86ab | 23a |
| 10⁻⁷M LCO | 86a | 86ab | 31a |
| 10⁻⁸M LCO | 84a | 86ab | 21a |

*Treatments followed by the same letter in a column are not significantly different when compared using Tukey's LSD test.

Plant height was observed at 7, 15 and 43 DAP (Table 9). A statistically significant increase in plant height versus the Untreated Control was observed at 7 DAP for the 10⁻⁶ and 10⁻⁸ NPG treatments, 15 DAP for the 10⁻⁷ and 10⁻⁸ treatments and all three NPG treatments at 43 DAP. NPG and LCO treatments exhibited comparable efficacy.

TABLE 9
Effect of NPG on Barley Plant Height (cm)

| Treatment | 7 DAP | 15 DAP | 43 DAP |
|---|---|---|---|
| Untreated | 4.8b | 11b | 64b |
| 10⁻⁶M NPG | 5.8a | 12ab | 71a |
| 10⁻⁷M NPG | 5.5ab | 14a | 71a |
| 10⁻⁸M NPG | 5.6a | 13a | 72a |
| 10⁻⁶M LCO | 5.9a | 13a | 71a |
| 10⁻⁷M LCO | 5.8a | 12ab | 72a |
| 10⁻⁸M LCO | 5.6a | 12ab | 72a |

*Treatments followed by the same letter in a column are not significantly different when compared using Tukey's LSD test.

Yield was determined by harvesting the entire plot and transforming the data to kg/ha (Table 10). Yields are not corrected for differences in emergence rates. NPG and LCO treatments did not provide greater yields that the Untreated Control. It is noteworthy, however, that yield benefits may be difficult to accurately determine with the small plots used in this study.

TABLE 10
Effect of NPG on Barley Yield (kg/ha)

| Treatment | Kg/Hectare |
|---|---|
| Untreated | 3439 |
| 10⁻⁶M NPG | 3184 |
| 10⁻⁷M NPG | 2504 |
| 10⁻⁸M NPG | 3043 |
| 10⁻⁶M LCO | 2535 |
| 10⁻⁷M LCO | 2380 |
| 10⁻⁸M LCO | 2418 |

### Example 5

### Effect of NPG on Plant Emergence, Vigor and Tillering on Spring Barley Grown Under Field Conditions in 2011

### Materials and Methods

A spring barley field trial was conducted to evaluate the effects of NPG on plant emergence and vigor in AC Metcalf spring barley (Hordeum vulgare) planted near Wetaskiwin, Alberta, Canada, in 2011. Seed treatments included an Untreated Control, 10⁻⁷ M NPG seed coating, 10⁻⁷ M NPG seed coating followed by an NPG foliar treatment (10⁻⁷ M NPG+Foliar), 10⁻⁶ M natural LCO, and 10⁻⁶ M commercial LCO plus a commercial rhizobia inoculant (LCO+RI) applied as a seed coating. The natural LCO was provided by Dr. Don Smith (McGill University, Montreal, Canada) and prepared as described in Example 2.

Seed coating was performed by injecting 7 mL of aqueous NPG solution per 100 g barley seed into the coating machine followed by treatment and drying. Upon completion of drying, NPG-coated seeds were placed in the coating machine a second time and injected with a maintenance fungicide treatment of 6.7 g/L tebuconazole and 222 g/L thiram at a rate of 225 mL/100 kg seed for protection against seed borne diseases. Foliar NPG was applied 47 days after planting. Plants were sprayed using a four-nozzle hollow-cone boom and CO₂ propellant at a speed of 10.8 km/h and 40 psi, with a mix size of 1.0 L and spray rate of 110 L/ha water volume. Seeds designated for use as the Untreated Control also received the fungicide maintenance treatment.

Treated seeds were sent to the DuPont Wetaskiwin, Alberta, Canada, Research Station and planted on May 19^{th} in a loam composed of 29% sand, 46% silt and 25% clay and 4.8% organic matter. The soil had a pH of 6.2 and cationic exchange capacity of 33. Barley was planted at a rate of 100 kg seeds/ha to a depth of 2.5 cm. Weeds were controlled using 60 grams ai/ha pinoxaden, 30 grams/ha thifensulfuron methyl, and 280 grams ai/ha 4-chloro-2-methylphenox acetic acid, 2-ethylhexyl ester. Adigor surfactant was used at a rate of 700 ml/hectare. All of the products used are considered industry standards and representative of what is used in commercial production.

The trial was conducted using a randomized complete block design with a plot size of 2 m by 6 m with 22.9 cm row spacing, 3.3 cm plant spacing and four replications. Height and yield data was not collected in this trial due to a large hailstorm on Jul. 18, 2011.

### Results

Emergence was observed at 11 and 14 DAP (Table 11). No statistically significant difference in emergence was observed among treatments at either time point.

TABLE 11
Effect of NPG on Barley Crop Emergence (plants/m row)

| Treatment | 11 DAP | 14 DAP |
|---|---|---|
| Untreated | 28a | 32.8a |
| 10⁻⁷M NPG Treatment #1 | 22.5a | 27.4a |
| 10⁻⁷M NPG + Foliar | 25a | 31.8a |
| 10⁻⁶M LCO | 31.1a | 34.0a |
| 10⁻⁶M LCO + RI | 28.3a | 31.4a |

Treatments followed by the same letter in a column are not significantly different when compared using Tukey's LSD test.

Crop vigor was observed 11 and 14 days DAP using the vigor scale described in Example 3 (Table 12).

No statistically significant difference in crop vigor was observed between treatments at 11 and 14 DAP.

TABLE 12
Effect of NPG on Barley Crop Vigor (1-5 scale)

| Treatment | 11 DAP | 14 DAP |
|---|---|---|
| Untreated | 3a | 4a |
| 10⁻⁷M NPG Treatment #1 | 3a | 4a |
| 10⁻⁷M NPG + Foliar | 3a | 4a |
| 10⁻⁶M LCO | 3a | 4a |
| 10⁻⁶M LCO + RI | 3a | 4a |

Treatments followed by the same letter in a column are not significantly different when compared using Tukey's LSD test.

Percent tillering (numbers of tillers per plant) was observed at 24 DAP (Table 13). No statistically significant difference was observed between treatments at 24 DAP.

TABLE 13
Effect of NPG on Barley Plant Tillers (%)

| Treatment | 24 DAP |
|---|---|
| Untreated | 94a |
| 10⁻⁷M NPG Treatment #1 | 83a |
| 10⁻⁷M NPG + Foliar | 95a |
| 10⁻⁶M LCO | 90a |
| 10⁻⁶M LCO + RI | 88a |

Treatments followed by the same letter in a column are not significantly different when compared using Tukey's LSD test.

### Example 6

### Effect of NPG on Plant Emergence, Vigor, Tillering, Biomass and Yield of Spring Wheat Under Field Conditions in 2011

### Materials and Methods

A field trial was conducted to evaluate the effects of NPG on plant emergence, vigor, tillering, biomass and yield in spring wheat (Triticum aestivum) planted near Breslau, Ontario, Canada, in 2011. Seed treatments included an Untreated Control, 10⁻⁷ M NPG applied as a seed treatment, 10⁻⁷ M NPG followed by two foliar NPG applications, 10⁻⁶ M natural LCO, and 10⁻⁶ M commercial LCO plus a commercial rhizobia inoculant (LCO+RI). The natural LCO was provided by Dr. Don Smith (McGill University, Montreal, Canada) and prepared as described in Example 2. Seed coating was performed by injecting 7 mL of aqueous NPG solution per 100 g wheat seed into a coating machine followed by treatment and drying. Upon completion of drying NPG-coated seeds were placed in the coating machine a second time and injected with a maintenance fungicide treatment of 6.7 g/L tebuconazole and 222 g/L thiram at a rate of 225 mL/100 kg seed for protection against seed borne diseases. Seed designated for use as the Untreated Check also received the fungicide maintenance treatment.

Treated seeds were sent to the DuPont Breslau, Ontario Research Station and planted on June 8^{th} in a loam composed of 34% sand, 48% silt and 18% clay and 2.9% organic matter. The soil had a pH of 7.4 and cationic exchange capacity of 19.3. Spring wheat was planted at a rate of 100 kg seeds/ha to a depth of 3 cm. Weeds were controlled using 8.79 wt % fenoxyprop-P-ethyl at a rate of 770 mL/ha and a mixture of 33.33 wt % thifensulfuron methyl and 16.67 wt % tribenuron methyl at a rate of 30 g/ha. Disease was controlled using 23.6 wt % pyraclostrobin fungicide at a rate of 0.4 L/ha. All of the products used are considered industry standards and representative of what is used in commercial production.

The trial was conducted using a randomized complete block design with a plot size of 2.5 m by 8 m with 17.8 cm row spacing, 3.3 cm plant spacing and four replications.

Foliar NPG applications were performed 36 and 49 days after planting. The treatments were sprayed using a 4 nozzle hollow-cone boom and CO₂ propellant at a speed of 4.5 km/h and 40 psi. The 36 day application utilized a mix size of 2.0 L and spray rate of 200 L/ha water volume. The 49 day application utilized a mix size of 3.0 L and spray rate of 300 L/ha water volume.

### Results

Percent emergence was observed at 13 and 35 DAP (Table 14). A non-statistically significant improvement in emergence was observed for the NPG, LCO+RI, and LCO treatments at 13 DAP. A performance distinction at 35 DAP was not possible because 100% germination had occurred in all treatments.

TABLE 14
Effect of NPG on Wheat Crop Emergence (%)

| Treatment | 13 DAP | 35 DAP |
|---|---|---|
| Untreated | 49a | 100a |
| 10⁻⁷M NPG | 53a | 100a |
| 10⁻⁷M NPG + Foliar | 53a | 100a |
| 10⁻⁶M LCO | 50a | 100a |
| 10⁻⁶M LCO + RI | 55a | 100a |

* Treatments followed by the same letter in a column are not significantly different when compared using Tukey's LSD test.

Crop vigor was observed 35, 40, and 52 DAP using the vigor scale described in Example 3 (Table 15). NPG treatments significantly increased vigor versus the Untreated Control at 40 DAP. This effect was not observed with the LCO and LCO+RI treatments. A non-statistically significant increase in vigor was observed for the NPG, LCO and LCO+RI treatments at 35 and 52 DAP.

TABLE 15
Effect of NPG on Spring Wheat Crop Vigor
Crop Vigor (1-5 scale)

| Treatment | 35 DAP | 40 DAP | 52 DAP |
|---|---|---|---|
| Untreated | 3.0a | 3.0a | 3.0a |
| 10⁻⁷M NPG | 3.4a | 3.5b | 3.4a |
| 10⁻⁷M NPG + | 3.6a | 3.5b | 3.5a |
| Foliar | | | |
| 10⁻⁶M LCO | 3.3a | 3.0a | 3.3a |
| 10⁻⁶M LCO + RI | 3.4a | 3.3a | 3.3a |

* Treatments followed by the same letter in a column are not significantly different when compared using Tukey's LSD test.

Tillering (number of tillers per plant) was observed at 35 days DAP (Table 16). A non-statistically significant improvement was observed for the NPG and LCO treatments versus the Untreated Control.

TABLE 16
Effect of NPG on Spring Wheat Tillering (tillers/plant)

| Treatment | 35 DAP |
|---|---|
| Untreated | 6.0a |
| 10⁻⁷M NPG | 9.0a |
| 10⁻⁷M NPG + Foliar | 8.0a |
| 10⁻⁶M LCO | 7.0a |
| 10⁻⁶M LCO + RI | 8.0a |

*Treatments followed by the same letter in a column are not significantly different when compared using Tukey's LSD test.

Plant Biomass was determined at 82 DAP by harvesting the entire aerial portion of the plants from each plot (Table 17). A non-statistically significant increase in biomass was determined for the NPG, LCO+RI, and LCO treatments compared to the Untreated Control.

TABLE 17
Effect of NPG on Spring Wheat Biomass (kg/plant)

| Treatment | 82 DAP |
|---|---|
| Untreated | 0.176a |
| 10⁻⁷M NPG | 0.190a |
| 10⁻⁷M NPG + Foliar | 0.198a |
| 10⁻⁶M LCO | 0.204a |
| 10⁻⁶M LCO + RI | 0.215a |

Treatments followed by the same letter in a column are not significantly different when compared using Tukey's LSD test.

Yield was determined by harvesting the entire 8 m of the plot and transforming the data to kg/ha (Table 18). Yields are not corrected for differences in emergence rates. A non-statistically significant improvement was observed for the NPG, LCO+RI, and LCO treatments compared to the Untreated Control.

TABLE 18
Effect of NPG on Spring Wheat Yield (kg/ha)

| Treatment | kg/ha |
|---|---|
| Untreated | 1113a |
| 10⁻⁷M NPG | 1100a |
| 10⁻⁷M NPG + Foliar | 1188a |
| 10⁻⁶M LCO | 1163a |
| 10⁻⁶M LCO + RI | 1288a |

Treatments followed by the same letter in a column are not significantly different when compared using Tukey's LSD test.

### Example 7

### Effect of NPG on Early Growth, Days to Maturity, Height and Yield of Canola Grown Under Field Conditions in 2010

### Material and Methods

A canola (Brassica napus) field trial was conducted to evaluate the effects of NPG on early growth, days to maturity, height and yield for Pioneer hybrid 45H28 planted at research sites near Carman, Manitoba, Canada, in the spring of 2010. Seed treatments included three concentrations of NPG and three concentrations of a natural LCO. The natural LCO was provided by Dr. Don Smith (McGill University, Montreal, Canada) and prepared as described in Example 2. The trial did not include an Untreated Control. Aqueous solutions of NPG and LCO were applied at 10⁻⁶M, 10⁻⁷ M and 10⁻⁸ M concentrations by soaking seeds in aqueous solutions of the respective treatments for 15 minutes followed by air drying on a tray. Control seeds were treated identically with the exception of being soaked in water without added NPG or LCO. Prior to NPG or LCO application, all seeds were treated with a liquid mixture of pesticides consisting of 20.7% thiamethoxam, 1.25% difenoconazole, 0.39% metalaxyl-M and 0.13% fludioxonil applied at a rate of 15 ml/kg of seed to minimize the effect of disease and insect damage.

The trial was conducted using a randomized complete block design with a plot size of 1.5 m by 6 m with a 19 cm row spacing and four replications. Canola was planted at a rate of 180 seeds/m⁻² to a depth of 1.25 cm. Border plots were utilized to minimize any border effect on seed yield. An herbicide mixture of sethoxidim (445 g ai/ha), ethametsulfuron-methyl (22 g ai/ha) and clopyralid (83 g ai/ha) was applied to plants at the 2-3 leaf stage to control all grassy and broadleaf weeds. Plants were also sprayed with boscalid (99 g ai/ha) at the 30% bloom stage to minimize the impact of sclerotinia stem rot on seed yield. Plants were harvested by straight cutting at physical maturity (87-88 days).

### Results

Early growth was scored on a 1-9 scale using a subjective evaluation of the ‘healthiness’ of plants and the soil surface area covered by their leaves when the plants are in the 4-6 leaf stage. This was done by observing a sufficient number of row/plots, including checks if possible, to establish a range from 1 (unhealthy/weak looking plants with small leaf coverage) to 9 (healthy/strong looking plants with large leaf coverage). No significant difference between treatments was observed on early growth.

Days to maturity was measured from time of planting to physiological maturity, which was recorded in days from planting until the seeds in the pod, one third of the way up the main raceme, had changed color to black in 50% of the plants in a given row or plot. No significant difference between treatments was observed in time to physiological maturity.

Plant height was measured at plant maturity. No significant difference between treatments was observed for plant height.

Yield was measured in bushels per acre of mature seed. Final harvest yield was corrected to 8% moisture. All three NPG treatments exhibited a non-statistically significant yield increase versus the LCO treatments (Table 19).

TABLE 19
Effect of NPG on Canola Yield (bushels/acre)

| Treatment | Yield (bu/a) |
|---|---|
| 10⁻⁶M NPG | 2531a |
| 10⁻⁷M NPG | 2517a |
| 10⁻⁸M NPG | 2609a |
| 10⁻⁶M LCO | 2380a |
| 10⁻⁷M LCO | 2365a |
| 10⁻⁸M LCO | 2310a |

Treatments followed by the same letter in a column are not significantly different when compared using Tukey's LSD test.

### Example 8

### Effect of NPG on Early Growth, Days to Maturity, Height and Yield of Canola Grown Under Field Conditions in 2011

### Materials and Methods

Three canola (Brassica napus) field trials were conducted in the spring of 2011 to evaluate the effects of NPG on early growth, days to maturity, height and yield for Pioneer hybrid 45H29 planted at research sites near Carman, Neepawa, and Treherne (Manitoba, Canada) in 2011. Seed treatments included an Untreated Control, 10⁻⁷ M NPG, and a mixture of a commercial LCO and rhizobia. An aqueous solution of NPG was applied at 0.25 L/100 kg seed by soaking the seeds in aqueous solutions of the respective treatments for 15 minutes followed by air drying on a tray. Untreated Control seeds were treated identically with the exception of being soaked in water without added NPG, LCO or rhizobia. The LCO/rhizobia mixture was applied to seeds at the manufacturers' recommended rates. Prior to NPG or LCO application, all seeds were treated with a liquid mixture of pesticides consisting of 20.7% thiamethoxam, 1.25% difenoconazole, 0.39% metalaxyl-M, and 0.13% fludioxonil applied at a rate of 15 mL/kg of seed to minimize the effect of disease and insect damage.

The trial was conducted using a randomized complete block design with a plot size of 1.5 m by 6 m with a 19 cm row spacing and four replications. Canola was planted at a rate of 180 seeds/m⁻² to a depth of 1.25 cm. Border plots were utilized to minimize any border effect on seed yield. An herbicide mixture of sethoxidim (445 g ai/ha), ethametsulfuron-methyl (22 g ai/ha) and clopyralid (83 g ai/ha) was applied to plants at the 2-3 leaf stage to control all grassy and broadleaf weeds. Plants were also sprayed with boscalid (99 g ai/ha) at the 30% bloom stage to minimize the impact of sclerotinia stem rot on seed yield. Plants were harvested by straight cutting at physical maturity (87-88 days). All results were averaged across locations for individual treatments.

### Results

Early growth was scored on a 1-9 scale using a subjective evaluation of the ‘healthiness’ of plants and the soil surface area covered by their leaves when the plants are in the 4-6 leaf stage. This was done by observing a sufficient number of row/plots, including checks if possible, to establish a range from 1 (unhealthy/weak looking plants with small leaf coverage) to 9 (healthy/strong looking plants with large leaf coverage). No significant difference on early growth was observed between treatments.

Days to maturity was measured from time of planting to physiological maturity, which was recorded in days from planting until the seeds in the pod, one third of the way up the main raceme, changed color to black in 50% of the plants in a given row or plot. No significant difference in time to physiological maturity was observed between treatments.

Plant height was measured at plant maturity. Plants treated with NPG averaged 114.2 cm in height versus 110.4 cm for the Untreated Controls and 112.5 cm for the rhizobia/LCO treatment. The differences were not statistically significant.

Yield was measured in bushels per acre of mature seed. Final harvest yield was corrected to 10% moisture. No significant difference in yield was observed between treatments.

### Example 9

### Effect of NPG on Yield of Corn Grown Under Field Conditions in 2011 Materials and Methods

The effect of NPG on corn (Zea mays) yield was evaluated in Pioneer seed treatment field trials during the 2011 growing season at research sites near Ames, Iowa, Bloomington, Ill., Champaign, Ill., and Ridgeway, Ill. Pioneer Hi-Bred hybrid P0902XR corn was planted in four row corn plots with 30 in row spacing and a plot length of 20 ft. At all research sites, each treatment was replicated four times with plant population data (number of plants per 2 middle plot rows) collected at the V4 corn growth stage, and corn grain yield data (bu/a) collected at harvest. Plots were managed by utilizing crop management practices common to each of the research site locations.

All seed treatments were composed of a standard fungicide seed treatment (FST) and insecticide seed treatment (IST) applied with and without NPG (Table 20). NPG was either applied in a slurry mixture (NPG-SL) with all other treatment components or as a pretreatment (NPG-PT) prior to the addition of the other seed treatment components. For both experimental treatments NPG was applied to corn seed using a 10⁻⁷ M solution.

TABLE 20
Seed treatment, application rates and application methods in corn.

| Treatment Number | Treatment Description | Application Method |
|---|---|---|
| 1 | FST/IST | Premixed components |
| | | applied as slurry |
| 2 | NPG-SL/FST/IST | Premixed components |
| | | applied as slurry |
| 3 | NPG-PT/FST/IST | NPG applied as seed |
| | | pretreatment. After seed |
| | | drying the remaining |
| | | premixed components |
| | | were applied as a slurry |

FST—fungicidal seed treatment (azoxystrobin, fludioxonil, mefenoxam, tebuconazole);
IST—insecticidal seed treatment (thiamethoxam)

### Results

Treatments were evaluated using plant population data collected from the V4 corn growth stage and corn grain yield at harvest. Experimental Treatments 2 and 3 did not provide a statistically significant yield improvement versus Treatment 1 (standard treatment) with respect to either absolute or corrected yield (bu/a) (Table 21).

TABLE 21
Corn plant population and yield response to seed treatments.

| | | | | Corrected |
|---|---|---|---|---|
| | | Plant Population | Yield | Yield* |
| Number | Treatment Code | (plants/acre) | (bu/a) | (bu/a) |
| 1 | FST/IST | 30,243 | 194.06a | 194.06a |
| 2 | NPG-SL/FST/IST | 28,974 | 183.08b | 184.38b |
| 3 | NPG-PT/FST/IST | 29,140 | 189.10ab | 190.24ab |

Data were analyzed using an analysis of variance for a randomized complete block design. Estimates were generated and significance declared at P ≦ 0.20.
*Yield for Treatments 2 and 3 corrected to plant population of Treatment 1.

NPG Treatments 2 and 3 did not provide a statistically significant yield improvement at any of the four locations (Table 22). NPG treatments did, however, exhibit a numerical yield advantage over Treatment 1 at the Ridgeway location, which was under the greatest environmental stress during the 2011 growing season.

TABLE 22
Corn grain yield response to NPG seed treatments across locations.

| Treatment | | Treatment | Estimated Yield |
|---|---|---|---|
| Number | Location | Description | (bu/a) |
| 1 | Ames, IA | FST/IST | 199.53a |
| 2 | | NPG-SL/FST/IST | 171.92b |
| 3 | | NPG-PT/FST/IST | 185.24ab |
| 1 | Bloomington, IL | FST/IST | 194.79a |
| 2 | | NPG-SL/FST/IST | 174.54b |
| 3 | | NPG-PT/FST/IST | 191.73ab |
| 1 | Champaign, IL | FST/IST | 207.13 |
| 2 | | NPG-SL/FST/IST | 203.28 |
| 3 | | NPG-PT/FST/IST | 200.07 |
| 1 | Ridgeway, IL | FST/IST | 174.80 |
| 2 | | NPG-SL/FST/IST | 180.83 |
| 3 | | NPG-PT/FST/IST | 179.42 |

Data were analyzed using an analysis of variance for a randomized complete block design. Estimates were generated and significance declared at P ≦ 0.20.

### Example 10

### Effect of NPG on the Yield of Soybeans Grown Under Field Conditions in 2011

### Materials and Methods

The effect of NPG on soybean (Glycine max) yield was evaluated in Pioneer seed treatment field trials during the 2011 growing season at research sites near Ames, Iowa, Bloomington, Ill., Champaign, Ill., Eldora, Iowa and Ridgeway, Ill. The field trials consisted of Pioneer 93Y70 brand soybeans planted at the Illinois research sites and Pioneer 92Y80 brand soybeans planted at the Iowa research sites. Soybeans were planted in four row plots with 30 in row spacing and a plot length of 20 ft. At all research sites, each treatment was replicated four times with soybean grain yield data (bu/a) collected at harvest. Plots were managed by utilizing crop management practices common to each of the research site locations. The trial included six treatments, which are summarized in Table 23. The pesticides, commercial rhizobia inoculant and commercial LCO were formulated into seed coatings at standard commercial application rates. NPG was either applied in a slurry mixture with all other treatment components (Treatment 5) or as a pretreatment to all other seed treatment components (Treatment 6). Both NPG treatments were applied to soybean seed using a 10⁻⁷ M solution.

TABLE 23
Seed treatment, application rates and application methods in soybeans.

| Treatment | Treatment | |
|---|---|---|
| Number | Description | Application Method |
| 1 | Untreated | None |
| 2 | RI | Premixed components applied as slurry |
| 3 | FST/IST/RI | Premixed components applied as slurry |
| 4 | FST/IST/LCO/RI | Premixed components applied as slurry |
| 5 | NPG-SL/FST/IST | Premixed components applied as slurry |
| 6 | NPG-PT/FST/IST | NPG was applied as seed pretreatment. |
| | | After seed drying the remaining |
| | | premixed components were applied as a |
| | | slurry |

FST—fungicidal seed treatment (metalaxyl + trifloxystrobin);
IST—insecticidal seed treatment (imidocloprid);
RI—rhizobia inoculant;
LCO—lipochitooligosaccharide

### Results

Treatments were evaluated using soybean grain yield harvest results (Table 24). NPG Treatments 5 and 6 provided a statistically equivalent yield to Treatments 3 and 4 and statistically greater yield than Treatments 1. Treatment 5 provided a statistically higher yield than Treatments 1 and 2, and the highest numerical yield among all treatments. These results indicate that NPG provides a yield benefit comparable to the commercial LCO product (Treatment 4).

TABLE 24
Soybean grain yield response to NPG seed treatments across locations.

| | | | Yield Advantage |
|---|---|---|---|
| Treatment | Treatment | | over Untreated |
| Number | Description | Yield (bu/a) | (bu/a) |
| 1 | Untreated | 62.25c | 0.00 |
| 2 | RI | 62.61bc | 0.36 |
| 3 | FST/IST/RI | 64.50ab | 2.25 |
| 4 | FST/IST/LCO/RI | 64.07ab | 1.82 |
| 5 | NPG-SL/FST/IST/RI | 65.02a | 2.77 |
| 6 | NPG-PT/FST/IST/RI | 62.14ab | −0.11 |

Data were analyzed using an analysis of variance for a randomized complete block design. Estimates were generated and significance declared at P ≦ 0.20.

A location-based yield analysis revealed statistically significant yield differences at the Ames, Bloomington and Ridgeway locations. At all three locations Treatment 5 statistically ranked among the highest yielding treatments and was equivalent to LCO-formulated Treatment 4. Treatment 5 also provided a statistically higher yield than Treatment 6 at the Ridgeway location, and a directional advantage at the Ames, Champaign and Eldora locations. These results demonstrate that the slurry seed application method utilized in Treatment 5 is more efficacious than NPG application as a pretreatment for soybeans (Treatment 6). This distinction was most apparent at the Ridgeway location, which was under the greatest environmental stress among the five locations during the 2011 growing season. These results indicate that Treatment 5 provides a relatively greater yield advantage under suboptimal growing conditions.

TABLE 25
Soybean grain yield Response to NPG seed treatments by location.

| Treatment | | Treatment | Estimated Yield |
|---|---|---|---|
| Number | Location | Description | (BPA) |
| 1 | Ames, IA | Untreated | 55.76c |
| 2 | | RI | 62.22a |
| 3 | | FST/IST/RI | 59.10ab |
| 4 | | FST/IST/LCO/RI | 59.78ab |
| 5 | | NPG-SL/FST/IST/RI | 60.45ab |
| 6 | | NPG-PT/FST/IST/RI | 58.82bc |
| | | | Average = 59.3 |
| 1 | Bloomington, IL | Untreated | 69.08ab |
| 2 | | RI | 65.35b |
| 3 | | FST/IST/RI | 69.51a |
| 4 | | FST/IST/LCO/RI | 66.43ab |
| 5 | | NPG-SL/FST/IST/RI | 66.92ab |
| 6 | | NPG-PT/FST/IST/RI | 67.38ab |
| | | | Average = 67.4 |
| 1 | Champaign, IL | Untreated | 62.04 |
| 2 | | RI | 63.82 |
| 3 | | FST/IST/RI | 64.44 |
| 4 | | FST/IST/LCO/RI | 63.78 |
| 5 | | NPG-SL/FST/IST/RI | 64.61 |
| 6 | | NPG-PT/FST/IST/RI | 60.76 |
| | | | Average = 63.2 |
| 1 | Eldora, IA | Untreated | 72.88 |
| 2 | | RI | 70.16 |
| 3 | | FST/IST/RI | 70.43 |
| 4 | | FST/IST/LCO/RI | 68.90 |
| 5 | | NPG-SL/FST/IST/RI | 72.44 |
| 6 | | NPG-PT/FST/IST/RI | 68.83 |
| | | | Average = 70.6 |
| 1 | Ridgeway, IL | Untreated | 51.47c |
| 2 | | RI | 51.51c |
| 3 | | FST/IST/RI | 59.03ab |
| 4 | | FST/IST/LCO/RI | 61.46a |
| 5 | | NPG-SL/FST/IST/RI | 60.69ab |
| 6 | | NPG-PT/FST/IST/RI | 54.90c |
| | | | Average = 56.5 |

Data were analyzed using an analysis of variance for a randomized complete block design. Estimates were generated and significance declared at P ≦ 0.20.

### Example 11

### Effect of NPG on Seed Germination Rates Under Cold Stress, Salt Stress and Non-Stressed Conditions

Materials and Methods

A series of Petri dish seed assays was conducted to evaluate the effects NPG on the seed germination rates of corn, soybean, and canola seeds subjected to cold stress, salt stress and non-stressed conditions (salt stress only for canola). Assays were performed with ten replications of ten seeds/plate (100 total seeds). NPG was applied to seeds at the specified concentrations prior to being placed in Petri plates. Seeds designated for Salt Stress Experiments 1 & 2 were placed in Petri dishes containing a 100 mM NaCl solution and incubated at 21° C.-22° C. in the dark. Cold stress Petri dishes were incubated at 15° C. in the dark. Untreated Controls were incubated at 21° C.-22° C. in the dark as were seeds utilized in the separately conducted non-stressed germination assays. Data was recorded as percent germination at designated times after plating. Statistical analyses were performed using one-way Anova and Kruskal-Wallis one-way analysis of variance on rank combined with Dunn's all pairwise multiple comparison procedure (α=0.05).

### Results

There was no statistically significant difference in germination rates for non-stressed corn seeds. The NPG treatment did, however, exhibit a directional increase in percent germination at all three time points (Table 26).

TABLE 26
Effect of NPG on Non-Stressed Corn Seed Germination (% germination
hours after plating).

| Treatment | 28 HAP | 34 HAP | 44 HAP |
|---|---|---|---|
| Untreated Control | 31a | 70a | 93a |
| 10⁻⁶M NPG | 34a | 80a | 100a |

There was no statistically significant difference in germination rates for corn seeds in Salt Stress Experiment 1. The 10⁻⁶ M NPG treatment showed a directional increase in percent germination at all three time points (Table 27).

TABLE 27
Effect of Salt Stress on NPG Corn Seed Germination (% germination
hours after plating). Experiment 1.

| Treatment | 30 HAP | 40 HAP | 48 HAP |
|---|---|---|---|
| Untreated Control | 25a | 64a | 91a |
| 10⁻⁶M D1 | 34a | 83a | 96a |
| 10⁻⁷M D1 | 23a | 68a | 91a |

There was no statistical significant difference in germination rates for corn seeds in Salt Stress Experiment 2 (Table 28). The 10⁻⁷ M NPG treatment exhibited a directional increase in percent germination at 32 HAP.

TABLE 28
Effect of Salt Stress on NPG Corn Seed Germination (% germination
hours after plating). Experiment 2.

| Treatment | 32 HAP | 42 HAP |
|---|---|---|
| Untreated Control | 26ab | 85a |
| 10⁻⁶M NPG | 20b | 90a |
| 10⁻⁷M NPG | 44a | 87a |

There was no statistically significant difference in germination rates for corn seeds subjected to cold stress (Table 29). Both NPG treatments exhibited a directional increase in percent germination at 48 HAP.

TABLE 29
Effect of Cold Stress on NPG Corn Seed Germination (% germination
hours after plating).

| Treatment | 48 HAP | 56 HAP | 65 HAP |
|---|---|---|---|
| Untreated Control | 11b | 55a | 100 |
| 10⁻⁶M NPG | 13ab | 52a | 100 |
| 10⁻⁷M NPG | 20ab | 52a | 100 |

There was no statistically significant difference in germination rates for non-stressed soybean seeds (Table 30).

TABLE 30
Effect of NPG on Non-Stressed Soybean Seed Germination (%
germination hours after plating).

| Treatment | 27 HAP | 34 HAP | 45 HAP |
|---|---|---|---|
| Untreated Control | 67a | 90a | 94a |
| 10⁻⁶M NPG | 70a | 93a | 97a |
| 10⁻⁶M NPG × 4 | 72a | 88a | 94a |
| Times | | | |

There was no statistically significant difference in germination rates for soybean seeds subjected to salt stress (Table 31). The NPG treatments did, however, exhibit a directional increases in germination rates at all time points.

TABLE 31
Effect of Salt Stress on NPG Soybean Seed Germination (% germination
hours after plating).

| Treatment | 27 HAP | 34 HAP | 45 HAP | 55 HAP | 65 HAP |
|---|---|---|---|---|---|
| Untreated | 14a | 51b | 80b | 90a | 94a |
| Control | | | | | |
| 10⁻⁶M NPG | 22a | 73ab | 83ab | 95a | 99a |
| 10⁻⁶M NPG × | 17a | 76ab | 89ab | 96a | 99a |
| 4 Times | | | | | |

There was no statistically significant difference in germination rates for soybean seeds subjected to cold stress (Table 31). Both NPG treatments exhibited a directional increase in percent germination at 30 and 44 HAP.

TABLE 32
Effect of Cold Stress on NPG Soybean Seed Germination (% germination
hours after plating).

| Treatment | 30 HAP | 36 HAP | 44 HAP | 50 HAP | 60 HAP |
|---|---|---|---|---|---|
| Untreated | 23a | 59a | 73a | 92a | 96a |
| Control | | | | | |
| 10⁻⁶M NPG | 26a | 58a | 80a | 90a | 96a |
| 10⁻⁶M NPG × | 29a | 58a | 81a | 90a | 97a |
| 4 Times | | | | | |
| 10⁻⁷M NPG | 23a | 51a | 84a | 91a | 95a |

There was no statistically significant difference in germination rates for canola seeds subjected to salt stress (Table 33). The 10⁻⁷ M NPG treatment exhibited a directional increase in percent germination at all time points.

TABLE 33
Effect of Salt Stress on NPG Canola Seed Germination (% germination
hours after plating).

| Treatment | 30 HAP | 39 HAP | 48 HAP |
|---|---|---|---|
| Untreated Control | 17.3ab | 63.4ab | 84a |
| 10⁻⁶M NPG | 14b | 67.3ab | 84a |
| 10⁻⁷M NPG | 22.7ab | 80.7a | 88.7a |

What is claimed is:

1. An agricultural composition comprising a compound represented by the general Formula 1, 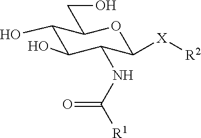
wherein R¹ is C₁-C₂₄ alkyl, C₇-C₂₄ alkaryl, C₆-C₂₄ aryl, C₂-C₂₄ monoalkenyl, C₄-C₂₄ dialkenyl or polyalkenyl, C₂-C₂₄ monoalkynyl, C₄-C₂₄ dialkynyl or polyalkynyl; R² is H, C₁-C₂₄ alkyl, C₇-C₂₄ alkaryl, or C₆-C₂₄ aryl, and X is O or S; wherein R¹ does not terminate with an aryl group when R¹ is mono-, di- or polyalkenyl or mono-, di- or polyalkynyl.

2. An agricultural composition of claim 1, wherein the compound is glucosamine amide N-palmitoleyl-D-glucosamine.

3. The agricultural composition of claim 1, wherein the agricultural composition is present in the formulation at a concentration of 10⁻⁵ M to 10⁻¹² M.

4. The agricultural composition of claim 2, wherein the agricultural composition is present in the formulation at a concentration of 10⁻⁵ M to 10⁻¹² M.

5. The agricultural composition of claim 2, wherein the agricultural composition is present in the formulation at a concentration of about 10⁻⁷ M.

6. The agricultural composition of claim 2, wherein the agricultural composition is applied to propagating material of the plant.

7. The agricultural composition of claim 6, wherein the agricultural composition is applied to propagating material of the plant to provide improved growth and yield under conditions of biotic or abiotic stress.

8. The agricultural composition of claim 6, wherein the propagating material is seed.

9. The agricultural composition of claim 8, wherein the formulation comprises one or more insecticides, fungicides, nematocides, bactericides, acaricides, entomopathogenic bacteria, viruses or fungi, growth regulators such as rooting stimulants, chemosterilants, repellents, attractants, pheromones, feeding stimulant and other signal compounds including, but not limited to, apocarotenoids, flavonoids, jasmonates and strigolactones.

10. The agricultural composition of claim 2, wherein the agricultural composition is applied to foliage.

11. The agricultural composition of claim 10, wherein the agricultural composition comprises one or more insecticides, fungicides, nematocides, bactericides, acaricides, entomopathogenic bacteria, viruses or fungi, growth regulators such as rooting stimulants, chemosterilants, repellents, attractants, pheromones, feeding stimulant and other signal compounds including, but not limited to, apocarotenoids, flavonoids, jasmonates and strigolactones.

12. The agricultural composition of claim 2, wherein the agricultural composition is applied to the soil either prior to or following planting plant propagating material.

13. The agricultural composition of claim 12, wherein the agricultural composition comprises one or more insecticides, fungicides, nematocides, bactericides, acaricides, entomopathogenic bacteria, viruses or fungi, growth regulators such as rooting stimulants, chemosterilants, repellents, attractants, pheromones, feeding stimulant and other signal compounds including, but not limited to, apocarotenoids, flavonoids, jasmonates and strigolactones.

14. A method for treating a plant, comprising applying an agricultural composition comprising a composition represented by the general Formula 1

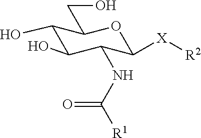

15. The method of claim 14, wherein the compound is glucosamine amide N-palmitoleyl-D-glucosamine.

16. The method of claim 15, wherein the agricultural composition is applied as a seed coating.

17. The method of claim 16, wherein the agricultural composition is a premixed slurry.

18. The method of claim 14, wherein the agricultural composition is applied to foliage.

19. The method of claim 14, wherein the agricultural composition is applied to soil either prior to or following planting plant propagating material.

20. The method of claim 14, wherein the agricultural composition is applied to a monocot.

21. The method of claim 14, wherein the agricultural composition is applied to a dicot.

22. The method of claim 14, wherein the agricultural composition is applied to a plant selected from a group consisting of barley, corn, millet, oats, rice, rye, sorghum, sugarcane and wheat.

23. The method of claim 14, wherein the agricultural composition is applied to a plant selected from a group consisting of canola, cotton, potatoes and soybeans.

24. The method according to claim 14, wherein the agricultural composition further comprises one or more insecticides, fungicides, nematocides, bactericides, acaricides, herbicides, plant nutrients, growth regulators such as rooting stimulants, chemosterilants, semiochemicals, repellents, attractants, pheromones, feeding stimulants, other biologically active compounds, microbial inocula or entomopathogenic bacteria, viruses or fungi.
